SEQUENCE LISTING (41 sequences)

SEQ ID NO: 1 - DNA, 250 bp, Homo sapiens
caggcatgaaccaccgcgcccggcctccaataaacatttattgggcacctacggtgttccaggcgctggtctaggcgttggggttacaacaacgcacaaaacataaattctgaagctgacacgctagctttaaacgttccttaaatatcttcatcctcaatttacggttgaggaaactgaggtgtagcgagaccttggaggtttccgaaatattattatggatggcaggtgtagagccctgccaacctct

SEQ ID NO: 2 - protein, 415 aa, Homo sapiens
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVTSQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGEGVLMEGGVKVREAVEEKNLGEAGRGLHACNPAFGRPRQAVT

SEQ ID NO: 3 - DNA, 3001 bp, Homo sapiens
actttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcacctcccagcccatccatgatagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgcggtgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcacgcagccccgagccctactcgactactgcgtcaccgcggacgccgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccttggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctcgcacgggcacccccagcctcaggtgactgacctgctgcctgcccccagcccccttcccggaccccctgtacagcgtccccacctatttcaaatcttatttaacaccccacacccacccctcagttgggacaaataaaggattctcatgggaaggggaggaccccgaattcct

SEQ ID NO: 4 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 5 - DNA, 6932 bp, Homo sapiens
gcatttaaaagacagcgtgagactcgcgccctccggcacggaaaaggccaggcgacaggtgtcgcttgaaaagactgggcttgtccttgctggtgcatgcgtcgtcggcctctgggcagcaggtttacaaaggaggaaaacgacttcttctagatttttttttcagtttcttctataaatcaaaacatctcaaaatggagacctaaaatccttaaagggacttagtctaatctcgggaggtagttttgtgcatgggtaaacaaattaagtattaactggtgttttactatccaaagaatgctaattttataaacatgatcgagttatataaggtataccataatgagtttgattttgaatttgatttgtggaaataaaggaaaagtgattctagctggggcatattgttaaagcatttttttcagagttggccaggcagtctcctactggcacattctcccattatgtagaatagaaatagtacctgtgtttgggaaagattttaaaatgagtgacagttatttggaacaaagagctaataatcaatccactgcaaattaaagaaacatgcagatgaaagttttgacacattaaaatacttctacagtgacaaagaaaaatcaagaacaaagctttttgatatgtgcaacaaatttagaggaagtaaaaagataaatgtgatgattggtcaagaaattatccagttatttacaaggccactgatattttaaacgtccaaaagtttgtttaaatgggctgttaccgctgagaatgatgaggatgagaatgatggttgaaggttacattttaggaaatgaagaaacttagaaaattaatataaagacagtgatgaatacaaagaagatttttataacaatgtgtaaaatttttggccagggaaaggaatattgaagttagatacaattacttacctttgagggaaataattgttggtaatgagatgtgatgtttctcctgccacctggaaacaaagcattgaagtctgcagttgaaaagcccaacgtctgtgagatccaggaaaccatgcttgcaaaccactggtaaaaaaaaaaaaaaaaaaaaaaaaaagccacagtgacttgcttattggtcattgctagtattatcgactcagaacctctttactaatggctagtaaatcataattgagaaattctgaattttgacaaggtctctgctgttgaaatggtaaatttattattttttttgtcatgataaattctggttcaaggtatgctatccatgaaataatttctgaccaaaactaaattgatgcaatttgattatccatcttagcctacagatggcatctggtaacttttgactgttttaaaaaataaatccactatcagagtagatttgatgttggcttcagaaacatttagaaaaacaaaagttcaaaaatgttttcaggaggtgataagttgaataactctacaatgttagttctttgagggggacaaaaaatttaaaatctttgaaaggtcttattttacagccatatctaaattatcttaagaaaatttttaacaaagggaatgaaatatatatcatgattctgtttttccaaaagtaacctgaatatagcaatgaagttcagttttgttattggtagtttgggcagagtctctttttgcagcacctgttgtctaccataattacagaggacatttccatgttctagccaagtatactattagaataaaaaaacttaacattgagttgcttcaacagcatgaaactgagtccaaaagaccaaatgaacaaacacattaatctctgattatttattttaaatagaatatttaattgtgtaagatctaatagtatcattatacttaagcaatcatattcctgatgatctatgggaaataactattatttaattaatattgaaaccaggttttaagatgtgttagccagtcctgttactagtaaatctctttatttggagagaaattttagattgttttgttctccttattagaaggattgtagaaagaaaaaaatgactaattggagaaaaattggggatatatcatatttcactgaattcaaaatgtcttcagttgtaaatcttaccattattttacgtacctctaagaaataaaagtgcttctaattaaaatatgatgtcattaattatgaaatacttcttgataacagaagttttaaaatagccatcttagaatcagtgaaatatggtaatgtattattttcctcctttgagttaggtcttgtgcttttttttcctggccactaaatttcacaatttccaaaaagcaaaataaacatattctgaatatttttgctgtgaaacacttgacagcagagctttccaccatgaaaagaagcttcatgagtcacacattacatctttgggttgattgaatgccactgaaacattctagtagcctggagaagttgacctacctgtggagatgcctgccattaaatggcatcctgatggcttaatacacatcactcttctgtgaagggttttaattttcaacacagcttactctgtagcatcatgtttacattgtatgtataaagattatacaaaggtgcaattgtgtatttcttccttaaaatgtatcagtataggatttagaatctccatgttgaaactctaaatgcatagaaataaaaataataaaaaatttttcattttggcttttcagcctagtattaaaactgataaaagcaaagccatgcacaaaactacctccctagagaaaggctagtcccttttcttccccattcatttcattatgaacatagtagaaaacagcatattcttatcaaatttgatgaaaagcgccaacacgtttgaactgaaatacgacttgtcatgtgaactgtaccgaatgtctacgtattccacttttcctgctggggttcctgtctcagaaaggagtcttgctcgtgctggtttctattacactggtgtgaatgacaaggtcaaatgcttctgttgtggcctgatgctggataactggaaaagaggagacagtcctactgaaaagcataaaaagttgtatcctagctgcagattcgttcagagtctaaattccgttaacaacttggaagctacctctcagcctacttttccttcttcagtaacaaattccacacactcattacttccgggtacagaaaacagtggatatttccgtggctcttattcaaactctccatcaaatcctgtaaactccagagcaaatcaagatttttctgccttgatgagaagttcctaccactgtgcaatgaataacgaaaatgccagattacttacttttcagacatggccattgacttttctgtcgccaacagatctggcaaaagcaggcttttactacataggacctggagacagagtggcttgctttgcctgtggtggaaaattgagcaattgggaaccgaaggataatgctatgtcagaacacctgagacattttcccaaatgcccatttatagaaaatcagcttcaagacacttcaagatacacagtttctaatctgagcatgcagacacatgcagcccgctttaaaacattctttaactggccctctagtgttctagttaatcctgagcagcttgcaagtgcgggtttttattatgtgggtaacagtgatgatgtcaaatgcttttgctgtgatggtggactcaggtgttgggaatctggagatgatccatgggttcaacatgccaagtggtttccaaggtgtgagtacttgataagaattaaaggacaggagttcatccgtcaagttcaagccagttaccctcatctacttgaacagctgctatccacatcagacagcccaggagatgaaaatgcagagtcatcaattatccattttgaacctggagaagaccattcagaagatgcaatcatgatgaatactcctgtgattaatgctgccgtggaaatgggctttagtagaagcctggtaaaacagacagttcagagaaaaatcctagcaactggagagaattatagactagtcaatgatcttgtgttagacttactcaatgcagaagatgaaataagggaagaggagagagaaagagcaactgaggaaaaagaatcaaatgatttattattaatccggaagaatagaatggcactttttcaacatttgacttgtgtaattccaatcctggatagtctactaactgccggaattattaatgaacaagaacatgatgttattaaacagaagacacagacgtctttacaagcaagagaactgattgatacgattttagtaaaaggaaatattgcagccactgtattcagaaactctctgcaagaagctgaagctgtgttatatgagcatttatttgtgcaacaggacataaaatatattcccacagaagatgtttcagatctaccagtggaagaacaattgcggagactacaagaagaaagaacatgtaaagtgtgtatggacaaagaagtgtccatagtgtttattccttgtggtcatctagtagtatgcaaagattgtgctccttctttaagaaagtgtcctatttgtaggagtacaatcaagggtacagttcgtacatttctttcatgaagaagaaccaaaacatcgtctaaactttagaattaatttattaaatgtattataactttaacttttatcctaatttggtttccttaaaatttttatttatttacaactcaaaaaacattgttttgtgtaacatatttatatatgtatctaaaccatatgaacatatattttttagaaactaagagaatgataggcttttgttcttatgaacgaaaaagaggtagcactacaaacacaatattcaatcaaaatttcagcattattgaaattgtaagtgaagtaaaacttaagatatttgagttaacctttaagaattttaaatattttggcattgtactaataccgggaacatgaagccaggtgtggtggtatgtgcctgtagtcccaggctgaggcaagagaattacttgagcccaggagtttgaatccatcctgggcagcatactgagaccctgcctttaaaaacaaacagaacaaaaacaaaacaccagggacacatttctctgtcttttttgatcagtgtcctatacatcgaaggtgtgcatatatgttgaatgacattttagggacatggtgtttttataaagaattctgtgagaaaaaatttaataaagcaacaaaaattactcttattcttcattgctttatttcaatgacattggatagtttagtcactcccagactctttccataccttcttaaagcctctcaaatattgaactacagtttatactccttcccataagatgcttcttcattgacacttgtagaacacggggtcaacacatcataaaatctattatggaatgcctgagacaagaatcaaacagtccctttagtaagtttgtttattcacttctctattgattcattcaagaagtctcatgccagccccacctattggaagaaggtctgagttttattcttatctctttggtattaattctgaaacttagaaagtacactggttagcaatgcttgggaccaacaggttgttctggtaaataaatctgtttcatattgtcagtgcaacaaaatgtccccctctgcattatgttattggtactcaacacgtccgagtcataactctgtcctttgcttcttatagaggtattaggtcttcaagagcagaagtaagactgtaatagggaatactcaggggaaggcaggcaaaggctagtcatctaaaccagttctagatgtctgtataggggcagatggctctgtaagggcagaagggaaagaccccttcataagggtcacagctgacaatcctataacaaaagacaggttaacaagagaaaaacttaacaaatttatttaatcacagatttacatcaccggggagccttcgtaatgaagatccaaaattacaggggaaactgtgcatttttatgcttaggtttgataatgaatggacagccctgaagaatagtgattggaaaaaaaggatatgatctaatgggaatagacacaggttggggacccagcaaggcctgtctgttcagattattcttggtctctgtgcagcattccttcctcctggatatagggcagggcctgtatgggatggggatattataacctgctatcaagcaaggtaggtcagagaatttatttatggccagctcttacatagttaggtgaggaaagattagagtactatctttaagatgtaagtctggcattgtggaaagatggttccagtttctatgacctaccttggggaagaggaattcaagtttctgtggcttgccttcagggagaatgaggctgagacaggagggcaggataacatcagagaaaaactttgcttctgaggccttcactttgggttttctgagccccaacatctgctagtgttgtaaagagaacaattagggaccaagtgaggggaggaaagaatccatctctgcattctgatgctgggagacttatttccttgaaatgcaattgattttgcctctgctaagaggctctgctggctacccatgtactagccagtgtcctgcatgggtgctaggctgaattatttgtaattgtgcttaggtgatttgtaactcaggtatagggtatttaaatagtaggcaccctttttgcaccatgtgtttttttttttatctagttcttgtatactacagataatatttgaactttgtcatctcactgtaaaacttttgttcatttctcattatggtaataaatagctattataaccaacccatttattcaaatatgttatttccctaagtgttattttgacattttgttttggaaaaaataaatcaccatagataataaaaaaaaaaaaaaaaaaaaaaa

SEQ ID NO: 6 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 7 - protein, 1605 aa, Homo sapiens
GAAGAAACCACCGGAAGGAACCATCTCACTGTGTGTAAACATGACTTCCAAGCTGGCCGTGGCTCTCTTGGCAGCCTTCCTGATTTCTGCAGCTCTGTGTGAAGGTGCAGTTTTGCCAAGGAGTGCTAAAGAACTTAGATGTCAGTGCATAAAGACATACTCCAAACCTTTCCACCCCAAATTTATCAAAGAACTGAGAGTGATTGAGAGTGGACCACACTGCGCCAACACAGAAATTATTGTAAAGCTTTCTGATGGAAGAGAGCTCTGTCTGGACCCCAAGGAAAACTGGGTGCAGAGGGTTGTGGAGAAGTTTTTGAAGAGGGCTGAGAATTCATAAAAAAATTCATTCTCTGTGGTATCCAAGAATCAGTGAAGATGCCAGTGAAACTTCAAGCAAATCTACTTCAACACTTCATGTATTGTGTGGGTCTGTTGTAGGGTTGCCAGATGCAATACAAGATTCCTGGTTAAATTTGAATTTCAGTAAACAATGAATAGTTTTTCATTGTACCATGAAATATCCAGAACATACTTATATGTAAAGTATTATTTATTTGAATCTACAAAAAACAACAAATAATTTTTAAATATAAGGATTTTCCTAGATATTGCACGGGAGAATATACAAATAGCAAAATTGAGGCCAAGGGCCAAGAGAATATCCGAACTTTAATTTCAGGAATTGAATGGGTTTGCTAGAATGTGATATTTGAAGCATCACATAAAAATGATGGGACAATAAATTTTGCCATAAAGTCAAATTTAGCTGGAAATCCTGGATTTTTTTCTGTTAAATCTGGCAACCCTAGTCTGCTAGCCAGGATCCACAAGTCCTTGTTCCACTGTGCCTTGGTTTCTCCTTTATTTCTAAGTGGAAAAAGTATTAGCCACCATCTTACCTCACAGTGATGTTGTGAGGACATGTGGAAGCACTTTAAGTTTTTTCATCATAACATAAATTATTTTCAAGTGTAACTTATTAACCTATTTATTATTTATGTATTTATTTAAGCATCAAATATTTGTGCAAGAATTTGGAAAAATAGAAGATGAATCATTGATTGAATAGTTATAAAGATGTTATAGTAAATTTATTTTATTTTAGATATTAAATGATGTTTTATTAGATAAATTTCAATCAGGGTTTTTAGATTAAACAAACAAACAATTGGGTACCCAGTTAAATTTTCATTTCAGATAAACAACAAATAATTTTTTAGTATAAGTACATTATTGTTTATCTGAAATTTTAATTGAACTAACAATCCTAGTTTGATACTCCCAGTCTTGTCATTGCCAGCTGTGTTGGTAGTGCTGTGTTGAATTACGGAATAATGAGTTAGAACTATTAAAACAGCCAAAACTCCACAGTCAATATTAGTAATTTCTTGCTGGTTGAAACTTGTTTATTATGTACAAATAGATTCTTATAATATTATTTAAATGACTGCATTTTTAAATACAAGGCTTTATATTTTTAACTTTAAGATGTTTTTATGTGCTCTCCAAATTTTTTTTACTGTTTCTGATTGTATGGAAATATAAAAGTAAATATGAAACATTTAAAATATAATTTGTTGTCAAAGTAAAAAAAAAAAAAAA

SEQ ID NO: 8 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 9 - DNA, 4753 bp, Homo sapiens
ctttggaaagtcccgtggaaatccccgggcctacaacccgcatacaactgaaacggggcaaagcagactgcgcagtctgcagtcttcgtggcgggccaagcgagcttggagcccgcgggggcggagcggtgagagcggccgccaagagagatcacacccccagccgaccctgccagcgagcgagcccgaccccaggcgtccatggagcgtcgcctccgcccggtccctgccccgacccccgcctgcggcgcgctcctgccttgaccaggacttgggactttgcgaaaggatcgcggggcccggagaggtaaccgccgcgcctcccggagaggtaaccgccgcgcctcccggagaggtgttggagagcacaatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggctaaccggaaacaggtgggtcacctcctgcaagaagtggggcctcgagctgtcagtcatcatggtgctatcctctgaacccctcagctgccactgcaacagtgggcttaagggtgtctgagcaggagaggaaagataagctcttcgtggtgcccacgatgctcaggtttggtaacccgggagtgttcccaggtggccttagaaagcaaagcttgtaactggcaagggatgatgtcagattcagcccaaggttcctcctctcctaccaagcaggaggccaggaacttctttggacttggaaggtgtgcggggactggccgaggcccctgcaccctgcgcatcaggactgcttcatcgtcttggctgagaaagggaaaagacacacaagtcgcgtgggttggagaagccagagccattccacctcccctcccccagcatctctcagagatgtgaagccagatcctcatggcagcgaggccctctgcaagaagctcaaggaagctcagggaaaatggacgtattcagagagtgtttgtagttcatggtttttccctacctgcccggttcctttcctgaggacccggcagaaatgcagaaccatccatggactgtgattctgaggctgctgagactgaacatgttcacattgacagaaaaacaagctgctctttataatatgcaccttttaaaaaattagaatattttactgggaagacgtgtaactctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagtgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagctatgtaataaaaaactatactctgtgttctgttaatgcctctgagtgtcctacctccttggagatgagatagggaaggagcagggatgagactggcaatggtcacagggaaagatgtggccttttgtgatggttttattttctgttaacactgtgtcctgggggggctgggaagtcccctgcatcccatggtaccctggtattgggacagcaaaagccagtaaccatgagtatgaggaaatctctttctgttgctggcttacagtttctctgtgtgctttgtggttgctgtcatatttgctctagaagaaaaaaaaaaaaggaggggaaatgcattttccccagagataaaggctgccattttgggggtctgtacttatggcctgaaaatatttgtgatccataactctacacagcctttactcatactattaggcacactttccccttagagccccctaagtttttcccagacgaatctttataatttctttccaaagataccaaataaacttcagtgttttcatctaattctcttaaagttgatatcttaatattttgtgttgatcattatttccattcttaatgtgaaaaaaagtaattatttatacttattataaaaagtatttgaaatttgcacatttaattgtccctaatagaaagccacctattctttgttggatttcttcaagtttttctaaataaatgtaacttttcacaagagtcaacattaaaaaataaattatttaagaacagaaaaaaaaaaaaaaaa

SEQ ID NO: 10 - DNA, 20 bp, Artificial Sequence
  Chemically Synthesized Primer
tccctggagaagagctacga

SEQ ID NO: 11 - DNA, 20 bp, Artificial Sequence
  Chemically Synthesized Primer
agcactgtgttggcgtacag

SEQ ID NO: 12 - DNA, 21 bp, Artificial Sequence
  Chemically Synthesized Primer
gaacgtcttgctcgagatgtg

SEQ ID NO: 13 - DNA, 21 bp, Artificial Sequence
  Chemically Synthesized Primer
ccagcaggtcagcaaagaatt

SEQ ID NO: 14 - DNA, 20 bp, Artificial Sequence
  Chemically Synthesized Primer
aatcccatcaccatcttcca

SEQ ID NO: 15 - DNA, 20 bp, Artificial Sequence
  Chemically Synthesized Primer
tggactccacgacgtactca

SEQ ID NO: 16 - DNA, 20 bp, Artificial Sequence
  Chemically Synthesized Primer
ccttggaagcaccatgtttg

SEQ ID NO: 17 - DNA, 20 bp, Artificial Sequence
  Chemically Synthesized Primer
ttgtgtggttcgaggcacat

SEQ ID NO: 18 - DNA, 22 bp, Artificial Sequence
  Chemically Synthesized Primer
gggaagaggagagagaaagagc

SEQ ID NO: 19 - DNA, 22 bp, Artificial Sequence
  Chemically Synthesized Primer
tccaggattggaattacacaag

SEQ ID NO: 20 - DNA, 21 bp, Artificial Sequence
  Chemically Synthesized Primer
tcctgatttctgcagctctgt

SEQ ID NO: 21 - DNA, 20 bp, Artificial Sequence
  Chemically Synthesized Primer
aatttctgtgttggcgcagt

SEQ ID NO: 22 - DNA, 20 bp, Artificial Sequence
  Chemically Synthesized Primer
tctgcttccaggtgacagtg

SEQ ID NO: 23 - DNA, 20 bp, Artificial Sequence
  Chemically Synthesized Primer
atcttgagctcggcagtgtt

SEQ ID NO: 24 - DNA, 18 bp, Artificial Sequence
  Chemically Synthesized Primer
agcatccttggggagagc

SEQ ID NO: 25 - DNA, 19 bp, Artificial Sequence
  Chemically Synthesized Primer
aggcagtcacctccacctc

SEQ ID NO: 26 - DNA, 20 bp, Artificial Sequence
  Chemically Synthesized Primer
gagaagccaatggagattgc

SEQ ID NO: 27 - DNA, 20 bp, Artificial Sequence
  Chemically Synthesized Primer
gacatcctgaaggtgctgct

SEQ ID NO: 28 - DNA, 20 bp, Artificial Sequence
  Chemically Synthesized Primer
cctctggtgaacctgcctac

SEQ ID NO: 29 - DNA, 20 bp, Artificial Sequence
  Chemically Synthesized Primer
taccctgcaccacagcaata

SEQ ID NO: 30 - DNA, 20 bp, Artificial Sequence
  Chemically Synthesized Primer
ggtattggtgagtcggatcg

SEQ ID NO: 31 - DNA, 20 bp, Artificial Sequence
  Chemically Synthesized Primer
tgctgcattgttcccataga

SEQ ID NO: 32 - DNA, 20 bp, Artificial Sequence
  Chemically Synthesized Primer
tcaagaccttcagctccaag

SEQ ID NO: 33 - DNA, 20 bp, Artificial Sequence
  Chemically Synthesized Primer
tgacgctgagcgtgaagaag

SEQ ID NO: 34 - DNA, 20 bp, Artificial Sequence
  Chemically Synthesized Primer
caacctctcgatcctgaagc

SEQ ID NO: 35 - DNA, 20 bp, Artificial Sequence
  Chemically Synthesized Primer
atcacgccttacccattgag

SEQ ID NO: 36 - DNA, 21 bp, Artificial Sequence
  Chemically Synthesized Primer
gaaaactttcgtcatactccg

SEQ ID NO: 37 - DNA, 21 bp, Artificial Sequence
  Chemically Synthesized Primer
gaaagtttgtgccttatggag

SEQ ID NO: 38 - DNA, 20 bp, Artificial Sequence
  Chemically Synthesized Primer
cacgagcaatgaagcaaatg

SEQ ID NO: 39 - DNA, 20 bp, Artificial Sequence
  Chemically Synthesized Primer
gtgcactggtgctttccttt

SEQ ID NO: 40 - DNA, 20 bp, Artificial Sequence
  Chemically Synthesized Primer
ctataatttgcgccgctgac

SEQ ID NO: 41 - DNA, 21 bp, Artificial Sequence
  Chemically Synthesized Primer
tttccttgggtcattgacttt

(19) United States
(12) Patent Application Publication
(10) Pub. No.: US 2021/0393597 A1
(43) Pub. Date: Dec. 23, 2021

(54) TARGETING THE TRANSCRIPTION FACTOR NF-KB WITH HARMINE

(71) Applicant: DANA-FARBER CANCER INSTITUTE, INC., Boston, MA (US)

(72) Inventors: David Frank, Lexington, MA (US); Erik Nelson, Cos Cob, CT (US)

(73) Assignee: DANA-FARBER CANCER INSTITUTE, INC., Boston, MA (US)

(21) Appl. No.: 17/289,816

(22) PCT Filed: Nov. 7, 2019
(86) PCT No.: PCT/US19/60260

§ 371 Date: Apr. 29, 2021

Related U.S. Application Data
(60) Provisional application No. 62/757,314, filed on Nov. 8, 2018.

(51) Int. Cl. A61K 31/437; A61K 31/475; A61K 31/505; A61K 31/538; A61K 31/675; A61K 38/17

(52) U.S. Cl. CPC A61K 31/437; A61K 31/475; A61K 38/1793; A61K 31/538; A61K 31/675; A61K 31/505

(57) ABSTRACT

The present invention relates to compositions and methods for treating cancer with harmine.

### RELATED APPLICATIONS

This application claims the benefit of priority under 35 U.S.C. § 119(e) to U.S. Provisional Application No. 62/757,314, filed Nov. 8, 2018, which is incorporated herein by reference in its entirety.

### GOVERNMENT LICENSE RIGHTS

This invention was made with government support under grant number R01-CA160979 awarded by the National Institutes of Health. The government has certain rights in the invention.

### BACKGROUND OF THE INVENTION

The transcription factor, nuclear factor kappa-light-chain-enhancer of activated B cells (NF-κB) regulates genes that control a range of cellular functions including proliferation, survival, and release of cytokines and chemokines. Consequently, increased or inappropriate activation of NF-κB is found frequently in cancer, inflammatory conditions, and auto-immune diseases. As such, prior to the invention described herein, there was a pressing need to develop compounds that directly inhibit NF-κB.

### SUMMARY OF THE INVENTION

The present invention is based upon the surprising discovery that harmine is an effective and specific inhibitor of NF-κB activity. Accordingly, as described herein, harmine and related compounds, such as harmol, are therapeutically effective against cancers and inflammatory diseases driven by increased NF-κB activity, both alone and in combination.

Provided are methods of inhibiting nuclear factor kappa-light-chain-enhancer of activated B cells (NF-κB) function or activity in a cell comprising contacting the cell with an agent derived from Peganum harmala (Syrian rue), or an analogue thereof, thereby inhibiting NF-κB function or activity in a cell. For example, the agent derived from Peganum harmala (Syrian rue) comprises harmine or harmol. In some cases, the method further comprises administering infliximab, adalimumab, certolizumab pegol, golimumab, or etanercept. For example, the NF-κB function or activity comprises NF-κB-dependent gene expression/transcriptional activity.

In one aspect, the harmine or harmol, or an analogue thereof, inhibits expression of a NF-κB target gene selected from the group consisting of baculoviral inhibitor of apoptosis protein repeat-containing protein 3 (BIRC3), interleukin 8 (IL-8), and tumor necrosis factor alpha-induced protein 3 (TNFAIP3; also known as A20).

In one aspect, the NF-κB function or activity in the cell is inhibited by 10%-100%, e.g., 15%, 20%, 25%, 30%, 35%, 40%, 45%, 50%, 55%, 60%, 65%, 70%, 75%, 80%, 85%, 90%, 95%, or 100%.

In some cases, the NF-κB inhibitor, e.g., harmine, harmine analogue, harmol, or harmol analogue is administered at a dose of 0.01 μM to 10 μM, e.g., 0.05 μM, 0.10 μM, 0.20 μM, 0.30 μM, 0.40 μM, 0.50 μM, 0.60 μM, 0.70 μM, 0.80 μM, 0.90 μM, 1.0 μM, 1.5 μM, 2.0 μM, 2.5 μM, 3 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, or 10 μM.

Also provided is a method for treating or preventing a hyperproliferative disorder or an inflammatory disease associated with aberrant NF-κB activity in a subject, e.g., a human subject, by administering to the subject a therapeutically effective amount of an agent derived from Peganum harmala (Syrian rue), or an analogue thereof, thereby treating or preventing the hyperproliferative disorder or inflammatory disease associated with aberrant NF-κB activity in the subject. Exemplary modes of administration of the NF-κB inhibitor include parental administration (e.g., subcutaneous and intravenous administration) and oral administration.

In some cases, the subject has been diagnosed with a hyperproliferative disorder or an inflammatory disease associated with aberrant NF-κB activity. In one aspect, the subject is identified as having elevated NF-κB activity, or the subject is identified as in need of inhibiting NF-κB activity. For example, NF-κB activity in the subject is 5% elevated, 10% elevated, 20% elevated, 30% elevated, 40% elevated, 50% elevated, 60% elevated, 70% elevated, 80% elevated, 90% elevated, or 100% elevated. The subject in need of inhibition of NF-κB will generally display enhanced NF-κB activity as described herein. It is readily apparent to one of ordinary skill in the art, based on the teachings herein, how to determine whether an individual has enhanced NF-κB activity.

For example, the agent derived from Peganum harmala (Syrian rue) comprises harmine, harmol, or an analogue thereof. In one example, the NF-κB inhibitor, i.e., an agent derived from Peganum harmala (Syrian rue), e.g., harmine, is administered soon after diagnosis with a hyperproliferative disorder, e.g., neoplasia, and before relapse of the disorder.

In some cases, the NF-κB inhibitor, i.e., the agent derived from Peganum harmala (Syrian rue), or analogue thereof are administered in combination. In other cases, one, two, three, or more agents derived from Peganum harmala (Syrian rue) or analogues thereof are administered.

In one example, the method further comprises administering infliximab, adalimumab, certolizumab pegol, golimumab, or etanercept. In one aspect, the NF-κB function or activity comprises NF-κB-dependent gene expression/transcriptional activity. In some cases, the harmine or harmol inhibits expression of a NF-κB target gene selected from the group consisting of BIRC3, IL-8, and TNFAIP3 (also known as A20).

For example, the NF-κB inhibitor, e.g., harmine, harmine analogue, harmol, or harmol analogue is administered at a dose of 0.01 μM to 10 μM, e.g., 0.05 μM, 0.10 μM, 0.20 μM, 0.30 μM, 0.40 μM, 0.50 μM, 0.60 μM, 0.70 μM, 0.80 μM, 0.90 μM, 1.0 μM, 1.5 μM, 2.0 μM, 2.5 μM, 3 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, or 10 μM.

In another example, harmine, harmine analogue, harmol, or harmol analogue is administered at a dose of about 50 mg to about 100 mg (e.g., about 50 mg, about 60 mg, about 70 mg, about 80 mg, about 90 mg, or about 100 mg) by mouth at least once daily (e.g., once daily, twice daily, three times daily, or four times daily). In another example, the harmine, harmine analogue, harmol, or harmol analogue is administered at a dose of about 1500 mg by mouth at least once daily (e.g., once daily, twice daily, three times daily, or four times daily).

In one example, the harmine, harmine analogue, harmol, or harmol analogue is administered once per month, once per week, once per day, every 12 hours, every 6 hours, every 4 hours, or every hour.

An exemplary hyperproliferative disorder comprises cancer. For example, the cancer comprises a solid tumor or a hematological cancer. Suitable solid tumors are selected from the group consisting of esophageal cancer, breast cancer, melanoma, colon cancer, stomach cancer, ovarian cancer, pancreatic cancer, lung cancer, hepatic cancer, head and neck cancer, prostate cancer, and brain cancer. For example, the solid tumor comprises triple negative breast cancer or high grade serous ovarian cancer. In some cases, the hematological cancer comprises leukemia, lymphoma, or multiple myeloma. For example, the leukemia is selected from the group consisting of acute lymphoblastic leukemia, acute myelogenous leukemia, chronic myelogenous leukemia, Hodgkin's disease, non-Hodgkin's lymphoma, T-cell lymphoma, B-cell lymphoma, and chronic lymphocytic leukemia.

In some cases, the harmine, harmine analogue, harmol, or harmol analogue inhibits or reduces the size of the cancer. For example, the harmine, harmine analogue, harmol, or harmol analogue inhibits or reduces the size of a tumor by 5%, 10%, 15%, 20%, 25%, 30%, 35%, 40%, 45%, 50%, 55%, 60%, 65%, 70%, 75%, 80%, 85%, 90%, 95%, or 100%.

In one example, the inflammatory disease associated with aberrant NF-κB activity comprises an autoimmune disease. NF-κB is constitutively active in many inflammatory diseases, such as inflammatory bowel disease, arthritis, sepsis, gastritis, asthma, and atherosclerosis, among others. Accordingly, suitable inflammatory diseases treatable by the methods described herein include inflammatory bowel disease, arthritis, sepsis, gastritis, asthma, and atherosclerosis. In other cases, the autoimmune disease is selected from the group consisting of celiac disease, diabetes mellitus type 1, Graves' disease, inflammatory bowel disease, multiple sclerosis, psoriasis, rheumatoid arthritis, and systemic lupus erythematosus.

In some cases, the methods further comprise administering a chemotherapeutic agent selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and vinorelbine.

In some cases, the methods further comprise administering a signal transducer and activator of transcription 3 (STAT3) inhibitor selected from the group consisting of pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, 3-(2-hydroxyphenyl)-3-phenyl-N,N-dipropylpropanamide, and derivatives or analogues thereof.

Also provided is an isolated ovarian cancer cell comprising a vector expressing a firefly luciferase reporter gene operably-linked to an NF-κB-dependent promoter. For example, the ovarian cancer cell comprises an OVCAR8 cell or an A2780 cell. In one aspect, the cell comprises a vector expressing Renilla luciferase operably linked to a constitutive promoter.

Methods of screening for a compound that inhibits NF-κB function and/or activity are carried out by providing one or more ovarian cancer cell(s) comprising a vector expressing a firefly luciferase reporter gene operably-linked to an NF-κB-dependent promoter; and contacting the cell(s) with a candidate compound, wherein a decrease in the level of NF-κB-dependent luciferase activity in the presence of the candidate compound as compared to the level of NF-κB-dependent luciferase activity in the absence of the candidate compound indicates that the candidate compound inhibits NF-κB function and/or activity.

In some cases, the methods further comprise contacting the cell with an agent that induces the function and/or activity of NF-κB prior to contacting the cell with a candidate compound. For example, the agent that induces the function and/or activity of NF-κB comprises TNFα.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “antineoplastic agent” is used herein to refer to agents that have the functional property of inhibiting a development or progression of a neoplasm in a human. Inhibition of metastasis is frequently a property of antineoplastic agents.

By “agent” is meant any small compound, antibody, nucleic acid molecule, or polypeptide, or fragments thereof.

By “agonist” is meant an agent capable of initiating the same reaction or activity typically produced by an endogenous substance. For example, an agonist binds to a receptor on a cell to initiate the same reaction or activity typically produced by the binding of the endogenous ligand.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Binding properties of an antibody to antigens, cells, or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist” is used in the broadest sense, and includes an agent that partially or fully blocks, inhibits, or neutralizes a biological activity of an epitope, polypeptide, or cell that it specifically binds. Methods for identifying antagonists may comprise contacting a polypeptide or cell specifically bound by a candidate antagonist with the candidate antagonist and measuring a detectable change in one or more biological activities normally associated with the polypeptide or cell.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., cancer, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion, e.g., a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

“Hybridization” means hydrogen bonding, which may be Watson-Crick, Hoogsteen, or reversed Hoogsteen hydrogen bonding, between complementary nucleobases. For example, adenine and thymine are complementary nucleobases that pair through the formation of hydrogen bonds.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography (HPLC). The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic complementary DNA (cDNA), a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “immunogenicity” is meant the ability of a particular substance, such as an antigen or epitope, to provoke an immune response in the body of a human or animal.

By “marker” is meant any protein or polynucleotide having an alteration in expression level or activity that is associated with a disease or disorder, e.g., neoplasia.

By “modulate” is meant alter (increase or decrease). Such alterations are detected by standard art-known methods such as those described herein.

The term, “normal amount” refers to a normal amount of a complex in an individual known not to be diagnosed with neoplasia. The amount of the molecule can be measured in a test sample and compared to the “normal control level,” utilizing techniques such as reference limits, discrimination limits, or risk defining thresholds to define cutoff points and abnormal values (e.g., for neoplasia). The “normal control level” means the level of one or more proteins (or nucleic acids) or combined protein indices (or combined nucleic acid indices) typically found in a subject known not to be suffering from neoplasia. Such normal control levels and cutoff points may vary based on whether a molecule is used alone or in a formula combining other proteins into an index. Alternatively, the normal control level can be a database of protein patterns from previously tested subjects who did not convert to neoplasia over a clinically relevant time horizon. In another aspect, the normal control level can be a level relative to a housekeeping gene.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia and of its evolution as well as the prospect of recovery as anticipated from usual course of the disease. Once the aggressiveness has been determined, appropriate methods of treatments are chosen.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “sample” as used herein refers to a biological sample obtained for the purpose of evaluation in vitro. Exemplary tissue samples for the methods described herein include tissue samples from tumors or the surrounding microenvironment (i.e., the stroma and/or infiltrating immune cells). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukapheresis). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 70%, at least 80%, at least 85%, at least 90%, at least 95%, or at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “subject” as used herein includes all members of the animal kingdom prone to suffering from the indicated disorder. In some aspects, the subject is a mammal, e.g., a human mammal or a non-human mammal. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1G is a series of immunoblots, bar graphs, and a line graph showing that STAT3 inhibition leads to increased NF-κB activity. FIG. 1A is an immunoblot and accompanying bar chart in which OVCAR8 cells were treated with Jak inhibitor 1 for 24 hours. STAT3 activation was measured by immunoblot (left) or qRT-PCR measurement of mRNA expression for NF-κB target genes (BIRC3, TNFAIP3, and IL-8) and the STAT3 target gene suppressor of cytokine signaling 3 (SOCS3; right). FIG. 1B is a bar chart wherein OVCAR8 cells were transfected with STAT3 or NF-κB dependent reporters and were treated with Jak Inhibitor 1 (Jak I) for 24 hours, after which luciferase activity was measured. FIG. 1C is a graph wherein Gene Set Enrichment Analysis (GSEA) was performed on gene expression data from cells treated with IL-6 and a Jak inhibitor as compared to IL-6 stimulation alone. GSEA demonstrates enrichment of an NF-κB signature in cells with STAT3 inhibition (Normalized Enrichment Score (NES) 1.99, p<0.001). FIG. 1D is an immunoblot and accompanying bar chart, wherein OVCAR8 cells were transfected with an siRNA targeting STAT3 (siRNA #3) and STAT3 expression was measured by immunoblot (left) and qRT-PCR (quantitative real-time polymerase chain reaction) measurement of NF-κB target gene expression (right). FIG. 1E is a bar chart, wherein OVCAR8 cells were transfected with siRNA to STAT3 or control and then transfected with STAT3 or NF-κB dependent luciferase reporters. Luciferase activity was measured 72 hours after STAT3 knockdown. FIG. 1F is a series of bar charts, wherein OVCAR8 cells were transfected with siRNA to STAT3 or control for 48 hours and then stimulated with TNFα to activate NF-κB for 1 hour. NF-κB target gene expression was measured by qRT-PCR. FIG. 1G is a bar chart wherein OVCAR8 cells were transfected with siRNA to STAT3 or control and then transfected with an NF-κB dependent reporter. The following day, cells were stimulated with TNFα for 5 hours and luciferase activity was measured.

FIG. 2A-FIG. 2C is a series of immunoblots and bar charts showing that p65 expression is required for increased NF-κB activity upon STAT3 inhibition. FIG. 2A is a photograph of an immunoglot and a bar chart, wherein OVCAR8 cells were transfected with the indicated siRNAs, after which STAT3 and p65 expression was measured by immunoblot (left). NF-κB target gene expression was measured by qRT-PCR (right). FIG. 2B is a photograph of an immunoblot, wherein following similar siRNA treatment, cells were fractionated into nuclear and cytoplasmic components. STAT3 and p65 was measured by immunoblot. PARP (poly ADP-ribose polymerase) served as a nuclear loading control and tubulin as a cytoplasmic loading control. FIG. 2C is a graph, wherein Hela-p65-EGFP cells were treated with Jak inhibitor 1 or TNF and nuclear translocation was measured. ****p<0.0001; ns, not significant.

FIG. 3A-FIG. 3G is a series of bar charts, immunoblots, and a line graph showing that RelB is a STAT3 target gene that mediates p65-dependent upregulation of NF-κB target genes upon STAT3 inhibition. FIG. 3A is a series of bar charts and a photograph of an immunoblot, wherein RELB mRNA expression was measured in OVCAR8 cells upon STAT3 inhibition with Jak inhibitor 1 (left) or siRNA to STAT3 (middle). RelB protein level was measured by immunoblot upon knockdown with the indicated siRNAs in OVCAR8 cells (right). FIG. 3B is a graph, wherein primary breast cancers were stratified based on staining for phosphorylated STAT3, and RELB mRNA expression was analyzed. FIG. 3C is a graph, wherein primary ovarian cancers were stratified based on RELB expression, and the presence of a STAT3 gene expression signature was determined by GSEA. A similar analysis was performed for primary multiple myeloma samples. FIG. 3D is a bar chart, wherein SKBR3 cells were stimulated with LIF to activate STAT3, and chromatin immunoprecipitation (ChIP) for STAT3 binding was performed to a candidate site in the RELB promoter, compared to a nonbinding region. FIG. 3E is a bar chart, where in OVCAR8 cells were transfected with siRNA to RelB or control, and NF-κB target gene expression was analyzed. FIG. 3F is a bar chart, wherein OVCAR8 cells were transfected with siRNA to RelB or control, then stimulated with TNF for 1 hour, and NF-κB target gene expression was analyzed. FIG. 3G is a bar chart, wherein OVCAR8 cells were transfected with siRNA to STAT3, followed by transfection with either GFP or RelB. NF-κB target gene expression was then measured by qRT-PCR. *p<0.05, **p<0.01, ***p<0.001 two-tailed t test.

FIG. 4A-FIG. 4F is a series of structures, bar graphs, line graphs, and an immunoblot showing that harmine inhibits NF-κB activity. FIG. 4A shows structures of the NF-κB inhibitor, harmine (left), and the inactive analogue, harmane (right). Also provided in FIG. 4A is the structure of harmol. FIG. 4B is a bar graph, wherein NF-κB-luc cells were treated with the indicated concentrations of harmine or harmane for 1 hour. Next, cells were stimulated with TNF for 5 hours after which luciferase activity was measured. FIG. 4C is a line graph, wherein INA6 myeloma cells were treated with the indicated concentrations of harmine or harmane for 72 hours and then, relative viable cell number was measured. FIG. 4D is a bar graph, wherein OVCAR8 cells were treated with harmine for 24 hours and then NF-κB target gene expression was measured by qRT-PCR. FIG. 4E is a photograph of an immunoblot, wherein OVCAR8 cells were treated with Jak inhibitor 1 or harmine for 24 hours and then stimulated with TNF. Cell fractionation was performed to separate the nuclear and cytoplasmic fractions. PARP and tubulin were the respective loading controls. FIG. 4F is a series of bar charts, wherein OVCAR8 cells were treated with harmine for 24 hours and then stimulated with TNF for 30 minutes. ChIP was performed and p65 and Pol II binding was measured at the indicated binding sites and expressed relative to a nonbinding control region. *p<0.05, **p<0.01, ***p<0.001 two tailed t test.

FIG. 5A-FIG. 5C is a series of bar charts and an immunoblot showing that a combination of STAT3 inhibitors and the NF-κB inhibitor, harmine, reduce the viability of cancer cells. FIG. 5A is a bar graph, wherein OVCAR8 cells were treated with the indicated inhibitors for 24 hours and NF-κB target gene expression was measured. FIG. 5B is a series of bar graphs, wherein INA6 (top), U266 (middle), and OVCAR8 (bottom) cells were treated with the indicated combination of inhibitors. Relative viable cell number was measured after 72 hours. Har, harmine; TG, TG101348. The concentrations used were as follows: INA6, 5 μM harmine, 0.25 μM Jak inhibitor 1; U266, 15 μM harmine, 3 μM TG101348; OVCAR8, 15 μM harmine, 5 μM pimozide. FIG. 5C is a photograph of an immunoblot and a bar graph, wherein INA6 cells were treated for 24 hours with the indicated inhibitors and apoptosis was measured by assessing PARP cleavage. Quantitation was performed using ImageJ software comparing the intensity of cleaved PARP to the full-length protein. *p<0.05, **p<0.01, ***p<0.001, **** p<0.0001 two tailed t test.

FIG. 6A-FIG. 6E is a series of bar charts and an immunoblot showing that STAT3 inhibition leads to NF-κB activation. FIG. 6A is a bar chart, wherein MDA-MB-468 breast cancer cells were treated with 1 μM Jak inhibitor 1 for 28 hours, after which NF-κB target gene expression was measured by qRT-PCR. FIG. 6B is a bar chart, wherein INA6 multiple myeloma (MM) cells were treated with Jak inhibitor 1 for 22 hours, after which NF-κB target gene expression was measured by qRT-PCR. FIG. 6C is an immunoblot and a bar chart, wherein A498 gastric cancer cells were treated with Jak inhibitor 1 for 24 hours, after which tyrosine phosphorylated STAT3 was measured by immunoblot (left) and expression of the NF-κB target gene, IL-8, was measured by RT-PCR (right). FIG. 6D is a bar chart, wherein OVCAR8 cells were treated with the indicated STAT3 inhibitor (i.e., nifu, nifuroxazide, pim, pimozide, pyr, or pyrimethamine) for 24 hours, after which NF-κB target gene expression was measured. FIG. 6E is a bar graph, wherein INA6 MM cells were treated with the indicated STAT3 inhibitor or dimethylsulfoxide (DMSO) control for 24 hours, after which NF-κB (BIRC3) and STAT3 (Bcl-x) target genes were measured by qRT-PCR.

FIG. 7A-FIG. 7F is a series of immunoblots and bar graphs showing that reduction of the expression of activated STAT3 leads to NF-κB activation. FIG. 7A is a photograph of an immunoblot, wherein OVCAR8 cells were transfected with siSTAT3 (pool) for the indicated times and analyzed by immunoblot with the indicated antibodies (top) and by qRT-PCR for the indicated NF-κB target genes (bottom). FIG. 7B is a bar graph, wherein MDA-MB-468 cells were transfected with siRNA to STAT3, and NF-κB target gene expression was analyzed. FIG. 7C is a bar graph, wherein A498 cells were transfected with siRNA to STAT3 and NF-κB target gene expression was measured. FIG. 7D is a bar graph, wherein OVCAR8 cells were transfected with the indicated siRNAs and NF-κB target gene expression was analyzed by qRT-PCR. FIG. 7E is a bar graph, wherein OVKATE cells were transfected with siRNA to STAT3 and analyzed by immunoblot with the indicated antibodies (top) and by qRT-PCR for the indicated NF-κB target genes (bottom). FIG. 7F is a photograph of an immunoblot and a bar graph, wherein OVCAR8 cells were stimulated with IFNγ for 1 hour and STAT1 activation was measured by immunoblot (above) and NF-κB target gene expression was measured by RT-PCR (bottom).

FIG. 8 is a bar graph showing that RelA (p65) upregulates NF-κB target gene expression. OVCAR8 cells were transfected with p65. 24 hours later, p65 expression was analyzed by immunoblot (left) and NF-κB target gene expression was measured by qRT-PCR and expressed relative to vector control (right).

FIG. 9 is an immunoblot and a bar chart showing that P65 expression is necessary for NF-κB gene expression upon STAT3 inhibition. OVCAR8 cells were transfected with siSTAT3 (siRNA #2) and/or sip65, and analyzed by immunoblot with the indicated antibodies (left) or by qRT-PCR for NF-κB target gene expression (right).

FIG. 10A-FIG. 10B is a series of photographs of immunoblots showing that STAT3 inhibition does not enhance nuclear localization of p65. FIG. 10A is a photograph of an immunoblot, wherein OVCAR8 cells were transfected with siRNA to STAT3 and then stimulated with TNF. Cellular fractionation was carried out, and nuclear p65 expression was measured by immunoblot. PARP served as the nuclear loading control. FIG. 10B is a photograph of an immunoblot, wherein HeLa-p65-EGFP cells were treated with Jak inhibitor 1 for 1 hour and analyzed by immunoblot for STAT3 tyrosine phosphorylation.

FIG. 11 is a bar graph showing that B-cell lymphoma 3-encoded protein (BCL3) inhibition enhances NF-κB target gene expression. OVCAR8 cells were transfected with siRNA to STAT3 or BCL3, and NF-κB target gene expression was analyzed by RT-PCR.

FIG. 12A-FIG. 12C is a series of graphs showing that STAT3 regulates RelB expression. FIG. 12A is a graph, wherein a potential STAT3 binding region was identified upstream of the RelB gene (labeled as Your Seq) using the UCSC genome browser (top). The DNA sequence of this region contains three canonical STAT3 binding site sites, which are highlighted in red (bottom) (SEQ ID NO: 1). FIG. 12B is a bar graph, wherein U266 cells treated with Jak inhibitor 1 for 3 h were analyzed by ChIP with antibodies to STAT3 and RNA polymerase (pol) II. Binding to the STAT3 site of RelB was measured by qPCR. FIG. 12C is a bar graph, wherein SKBR3 cells were transfected with siRNA to STAT3 or RelB, and expression of the NF-κB target gene, IL-8, was measured by qRT-PCR.

FIG. 13A-FIG. 13C is a series of bar graphs showing that harmine is an NF-κB inhibitor. FIG. 13A is a bar graph, wherein the Prestwick chemical library was screened for NF-κB inhibitors using an NF-κB-dependent reporter and counterscreened using a STAT3 dependent reporter. Harmine was identified as an inhibitor of NF-κB-dependent luciferase with little effect on STAT3. Harmane, a structural variant of harmine, does not inhibit NF-κB or STAT3 activity. FIG. 13B is a bar graph, wherein U266 cells (which display constitutive STAT3 activation) and MA/11.S cells (which lack STAT3 activation), both of which contain constitutively active NF-κB, were treated with the indicated doses of harmine. Cell viability was measured after 72 hours of treatment. FIG. 13C is a bar graph, wherein OVCAR8 cells were treated for 24 hours with harmine or INDY and NF-κB target gene expression was analyzed. *p<0.05, **p<0.01, ***p<0.001,****p<0.0001, two-tailed t-test.

### DETAILED DESCRIPTION OF THE INVENTION

The invention is based, at least in part, upon the identification that harmine is an effective and specific inhibitor of NF-κB. Also described herein are results demonstrating that signal transducer and activator of transcription 3 (STAT3) modulates NF-κB activity by upregulating negative regulators of NF-κB. Additionally, the rationale for combination therapy targeting oncogenic transcription factors is described in detail below.

While many strides have been made in developing anti-cancer agents targeting oncogenic pathways, prior to the invention described herein, clinical benefit from these drugs had been modest. One factor limiting the effectiveness of signaling inhibitors is the activation of compensatory pathways. The transcription factor, STAT3, is activated inappropriately in a wide range of human cancers. Therefore, a key biological and translational question is whether inhibition of STAT3 will lead to activation of other oncogenic pathways. As described in detail below, inhibition of STAT3 results in upregulation of NF-κB transcriptional activity mediated by p65 (RelA). This response involves RelB, an NF-κB family member that can function as a negative regulator of p65, which is a direct target gene of STAT3. As also described herein, decreased expression of RelB in the setting of STAT3 inhibition leads to increased expression of a cohort of NF-κB target genes. Given this reciprocal relationship between STAT3 inhibition and NF-κB activity, as described in detail herein, it was of interest to identify inhibitors of NF-κB that could be used in therapeutic combinations with STAT3 inhibitors. Using a cell-based screen, the natural product, harmine, was identified as an NF-κB inhibitor, and it was determined that harmine synergistically kills cancer cells in combination with STAT3 inhibitors. These findings suggest that the combination of STAT3 and NF-κB inhibitors may be an important therapeutic strategy in cancer.

Signal transducers and activators of transcription (STATs) are transcription factors that regulate genes involved in critical cellular processes such as proliferation, survival, and self-renewal. While the activation of STATs is tightly regulated in normal cells, STATs become activated constitutively in many cancers where they drive expression of key target genes underlying the malignant phenotype (Frank D A. 2007 Cancer Lett, 251(2):199-210). There are seven members of the STAT family, with STAT3 being activated most commonly in cancers, including a wide range of hematopoietic cancers such as multiple myeloma, as well as solid tumors such as breast cancer, ovarian cancer, and gastric cancer (Catlett-Falcone et al., 1999 Immunity, 10:105-15; Bromberg J. 2000 Breast Cancer Res, 2(2):86-90; Kanda et al., 2004 Oncogene, 23(28):4921-9; Huang et al., 2000 Gynecol Oncol, 79(1):67-73).

Tumor cells are often dependent on continual STAT3 activation for their survival, whereas normal cells can tolerate disruption in STAT3 activity with few deleterious effects (Frank D A. 2007 Cancer Lett, 251(2):199-210). Consequently, there has been a great interest in developing drugs that target STAT3, and its major upstream regulators such as Jak family kinsases (Walker S R. and Frank D A. 2012 JAKSTAT, 1(4):292-9; Johnson et al., 2018 Nat Rev Clin Oncol, 15(4): 234-248). STAT3 inhibitors are currently in clinical trials for cancer and a variety of inflammatory conditions (Hubbard et al., 2017 Drugs, 77(10):1091-103; Punwani et al. 2015 Br J Dermatol, 173(4):989-97). However, many other inhibitors of oncogenic signaling pathways have shown only limited activity, often because of the compensatory activation of parallel signaling pathways, suggesting that a similar phenomenon may also occur with STAT3 inhibitors (Shi R et al., 2014 Pharmazie, 69(5):346-52; Talati C and Pinilla-Ibarz J. 2018 Curr Opin Hematol, 25(2):154-61; Herr et al., 2018 Oncogene, 37(12):1576-93).

One transcription factor pathway that has cross-talk with STAT3 is NF-κB, which is also activated frequently in cancer and in inflammatory conditions, and consists of five family members, RelA (p65), RelB (p68), c-Rel, p50, and p52 (Grivennikov S I and Karin M. 2010 Cytokine Growth Factor Rev, 21(1):11-9; Karin et al., 2002 Nat Rev Cancer, 2(4):301-10). These components are retained in an inactive state in the cytoplasm as either full-length proteins (p105 for p50, p100 for p52) or in complexes with IKB proteins (Wang et al., 2002 Int Immunopharmacol, 2(11):1509-20; Sun S C. 2012 Immunol Rev, 246(1):125-40). Activation of NF-κB signaling requires upstream signals to target the IKB proteins for degradation or induce cleavage of full-length proteins, with subsequent translocation of the transcriptionally active proteins into the nucleus (Magnani et al., 2000 Curr Drug Targets, 1(4):387-99). NF-κB proteins act as dimers, such as RelA/p50 heterodimers, to regulate genes involved in survival, migration, and other phenotypes (Lee J I and Burckart G J. 1998 J Clin Pharmacol, 38(11):981-93).

Given the importance of understanding potential modes of resistance to STAT3 inhibition, the activity of NF-κB upon treatment of cancer cells with STAT3 inhibitors was analyzed herein. As described in detail below, STAT3 inhibition resulted in p65-dependent activation of NF-κB target genes. Additionally, a small molecule was identified that inhibits p65 activity in cancer cells and shows efficacy in combination with STAT3 inhibitors.

While inhibition of STAT3 and its upstream kinases shows promise for the treatment of many cancers, compensatory regulatory mechanisms may limit their efficacy. Here, one mechanism was identified limiting the efficacy of Janus kinase (JAK)-STAT inhibitors via the compensatory upregulation of NF-κB activity. Moreover, as set forth in detail below, the NF-κB subunit, RelB, was identified as a direct STAT3 target gene, which affects regulation of p65 specific target genes.

Many cancers contain activation of both STAT3 and NF-κB, and there are complex positive and negative interactions between these two signaling pathways. For example, the cytokine interleukin (IL)-6 is a known target gene of NF-κB, which leads to STAT3 activation (Libermann T A and Baltimore D. 1990 Mol Cell Biol, 10(5):2327-34; Nakajima et al., 1996 Embo J, 15(14):3651-8). On the other hand, STAT3 increases expression of the microRNA miR-146b, which can down regulate NF-κB activity (Xiang, et al., 2016 Blood, 128: 1845-1853). In fact, this negative feedback mechanism is frequently suppressed in primary human cancers. In cancers that contain activation of both STAT3 and NF-κB, the level of RelB expression may maintain a balance of some p65-dependent activity to promote cancer cell growth and survival. However, inhibition of STAT3 reduces the level of RelB and possibly other negative regulators, thus disrupting this balance, allowing NF-κB to become more transcriptionally active. Additionally, in the setting of inflammation and increased expression of inflammatory cytokines such as TNF, loss of RelB may result in superactivation of NF-κB.

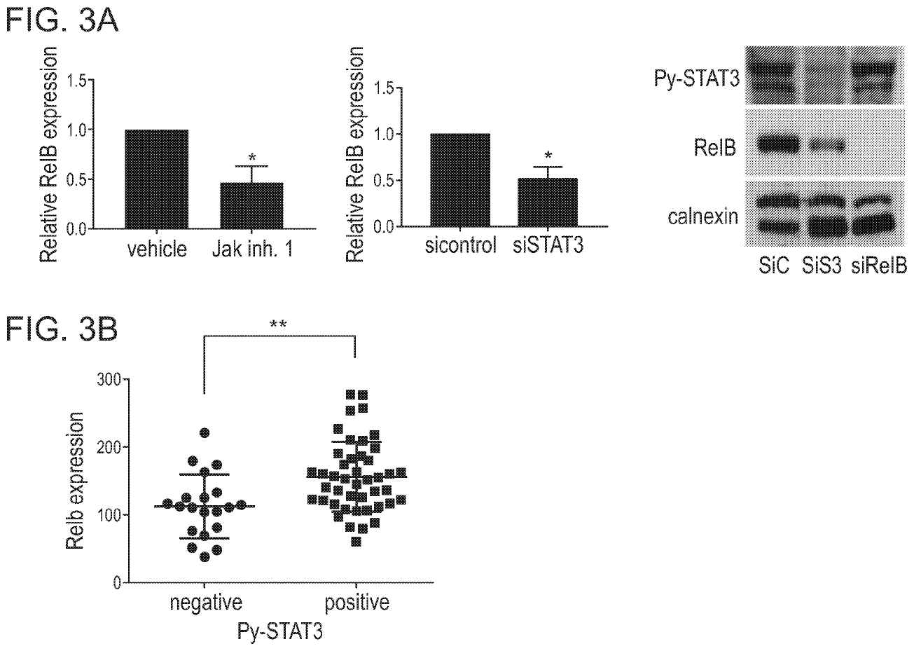

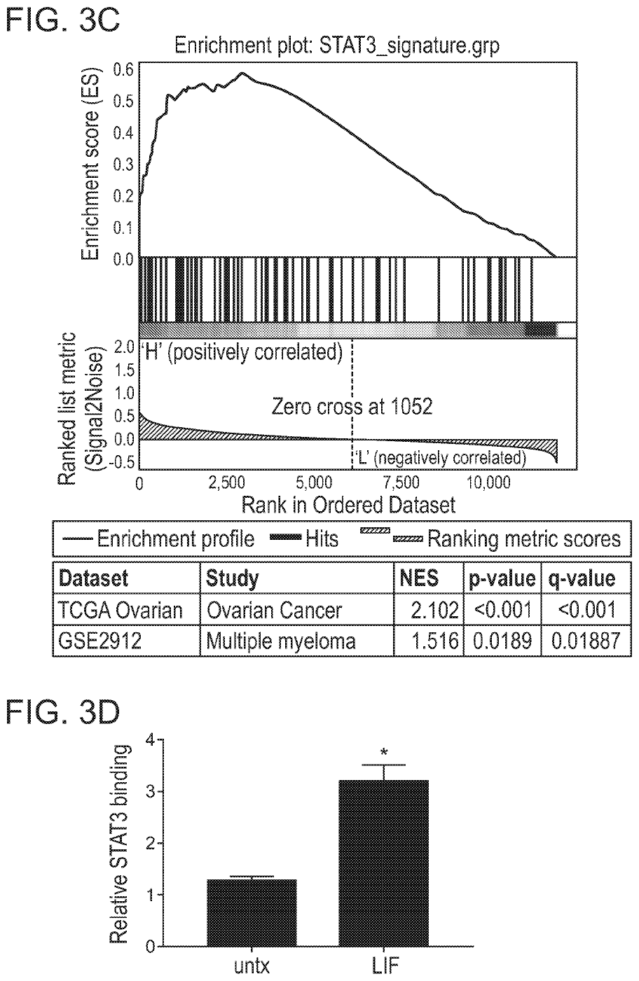

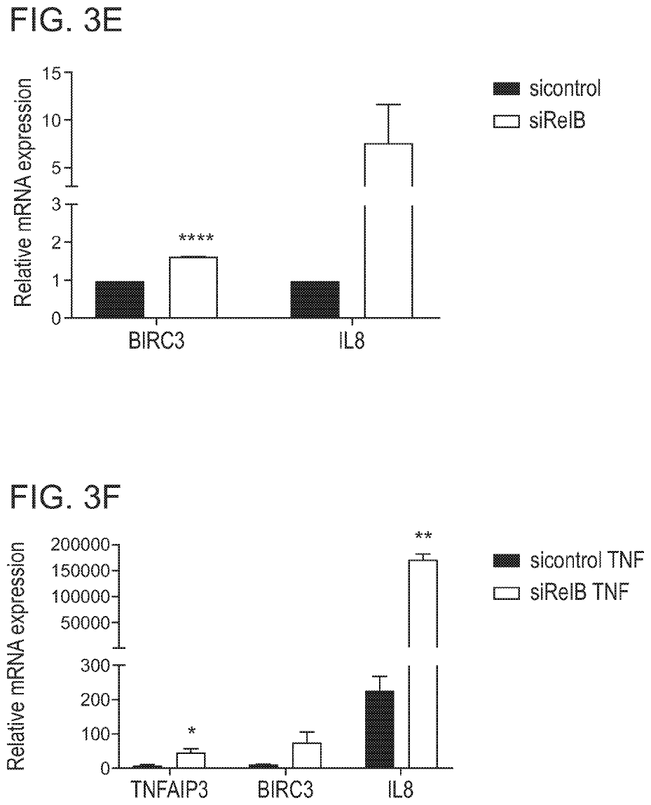

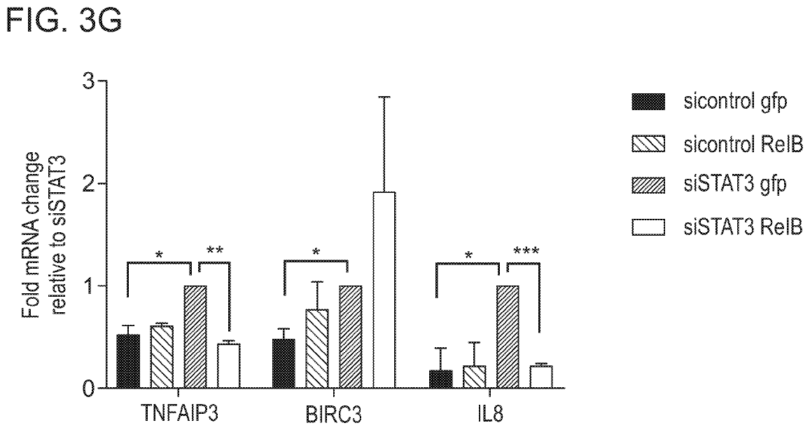

As described in the examples below, increased NF-κB target gene expression was not seen when RelB was re-expressed in the setting of STAT3 reduction. This suggests that RelB plays a pivotal role in mediating the effects between p65 and STAT3 on at least some target genes. Interestingly, the gene, BIRC3, was not rescued by RelB expression (FIG. 3G). In fact, RelB expression promoted the expression of BIRC3 mRNA regardless of STAT3 expression. In other systems, RelB has also been shown to upregulate BIRC3 (Cormier et al., 2013 PLoS One, 8(3):e59127). However, reduction of RelB expression also increased BIRC3 expression in OVCAR8 cells (FIG. 3D). This suggests that the effects of RelB on the regulation of BIRC3 expression is more complex than its effects on TNFAIP3 and IL8.

Another mechanism by which STAT3 can affect NF-κB is through modulation of the localization of p65. For example, in the context of K-RAS mutations, STAT3 has been reported to retain p65 in the cytoplasm (Grabner et al., 2015 Nat Commun, 6:6285). In addition, treatment of cells with JSI-1 24, which can inhibit STAT3, also led to translocation of p65 into the nucleus (McFarland et al., 2013 Mol Cancer Res, 11(5):494-505). Thus, STAT3 inhibition can possibly result in increased p65 in the nucleus; however, using a variety of STAT3 inhibitors in multiple cell lines, changes in p65 nuclear localization was not observed. Nonetheless, STAT3 may regulate p65 activity by other mechanisms, depending on the cellular context.

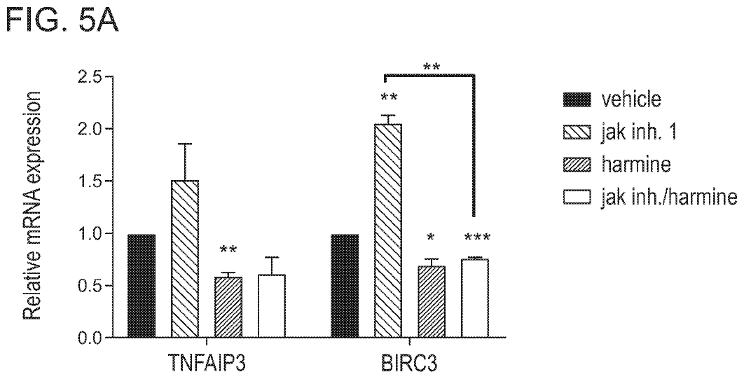

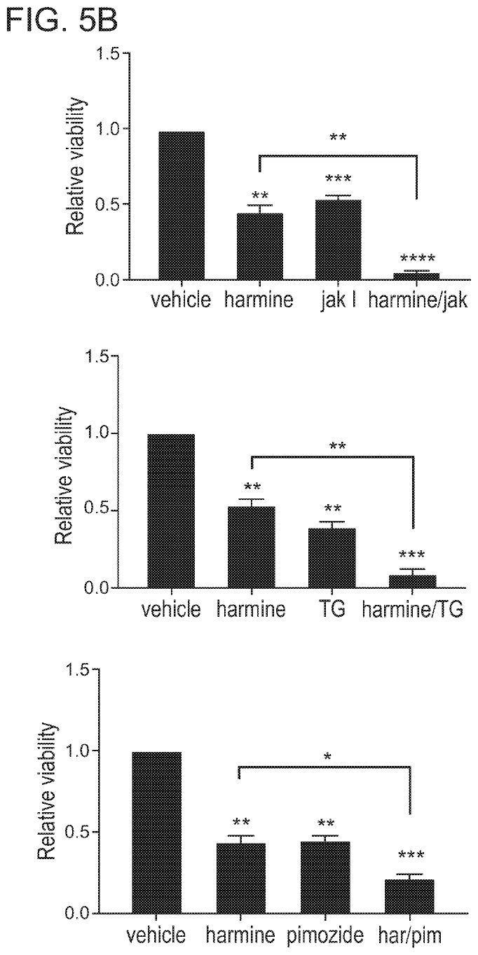

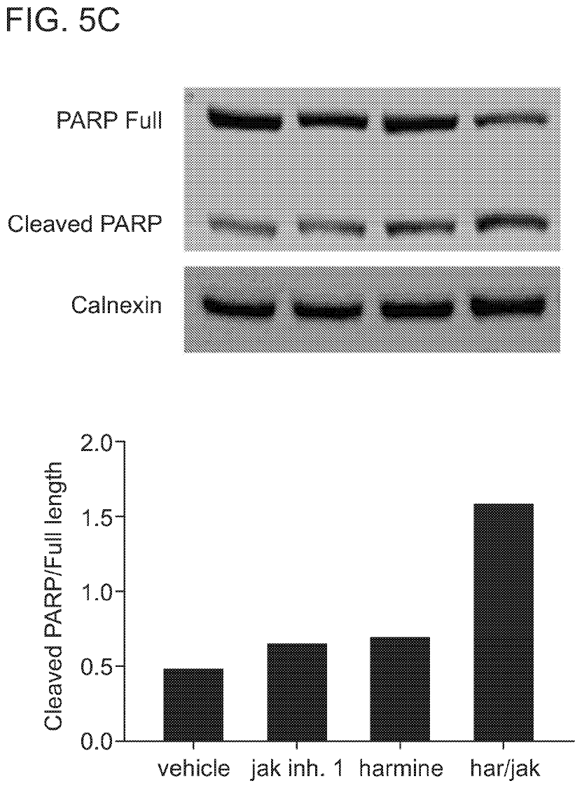

As STAT3 and NF-κB are activated in many cancers, and it appears that STAT3 and NF-κB maintain a balance based on expression of target genes, targeting both factors may be necessary for optimal cancer treatment. Inhibition of STAT3 alone can result in loss of NF-κB negative regulators leading to enhanced NF-κB activity. Therefore, as described herein, it is useful to target these pathways simultaneously, such as with a STAT3 inhibitor and the NF-κB inhibitor, harmine, as these drugs combined synergistically to enhance loss of viability (FIG. 5). As described in the Examples below, this approach would be of potential use in a variety of cancers characterized by activation of both pathways, including triple negative breast cancer, high grade serous ovarian cancer, gastric cancer, and multiple myeloma.

### Signal Transducers and Activators of Transcription (STAT) Molecules

Signal transducers and activators of transcription (STATs) are a family of transcription factors that play important roles in a range of cellular functions. STATs reside in the cytoplasm under basal conditions. Upon activation by tyrosine phosphorylation, STATs dimerize, translocate to the nucleus, bind to DNA, and regulate transcription of target genes that regulate cellular functions such as survival, proliferation, and differentiation (Darnell, J. E., Jr., 1997 Science 277, 1630-1635). Under physiological conditions, STATs are activated only transiently. By contrast, in many forms of cancer, STAT family members are activated constitutively and drive the expression of genes underlying malignant cellular behavior.

Specifically, members of the STAT protein family are intracellular transcription factors that mediate many aspects of cellular immunity, proliferation, apoptosis, and differentiation. There are seven mammalian STAT family members that have been identified: STAT1, STAT2, STAT3, STAT4, STAT5 (STAT5A and STAT5B), and STATE. STAT proteins are primarily activated by membrane receptor-associated Janus kinases (JAK). Dysregulation of the JAK/STAT pathway is frequently observed in primary tumors and leads to increased angiogenesis, enhanced survival of tumors, and immunosuppression. STAT proteins are involved in the development and function of the immune system and play a role in maintaining immune tolerance and tumor surveillance.

STAT proteins are present in the cytoplasm of cells under basal conditions. When activated by tyrosine phosphorylation, STAT proteins form dimers and translocate to the nucleus where they can bind specific nine-base-pair sequences in the regulatory regions of target genes, thereby activating transcription. A variety of tyrosine kinases, including polypeptide growth factor receptors, Src family members, and other kinases can catalyze this phosphorylation. While tyrosine phosphorylation is essential for their activation, STAT proteins can also be phosphorylated on unique serine residues. Although this is not sufficient to induce dimerization and DNA binding, STAT serine phosphorylation modulates the transcriptional response mediated by a tyrosine-phosphorylated STAT dimer, and may mediate distinct biological effects (Zhang X, et al. Science 1995; 267:1990-1994; Wen Z, et al. Cell 1995; 82:241-250; Kumar A, et al. Science 1997; 278:1630-1632). STAT proteins function inappropriately in many human malignancies (Alvarez J V, et al., Cancer Res 2005; 65(12):5054-62; Frank D A, et al. Cancer Treat. Res. 2003; 115:267-291; Bowman T, et al. Oncogene 2000; 19(21):2474-88).

### Nuclear Factor Kappa-Light-Chain-Enhancer of Activated B Cells (NF-κB)

NF-κB is a protein complex that controls transcription of DNA, cytokine production and cell survival. NF-κB is present in almost all animal cell types and is involved in cellular responses to stimuli such as stress, cytokines, free radicals, heavy metals, ultraviolet irradiation, and bacterial or viral antigens. NF-κB is also involved in regulating the immune response to infection. Incorrect regulation and/or aberrant expression of NF-κB has been linked to cancer, inflammatory and autoimmune diseases, septic shock, viral infection, and improper immune development.

All proteins of the NF-κB family share a “Rel” homology domain in their N-terminus. A subfamily of NF-κB proteins, including RelA, RelB, and c-Rel, have a transactivation domain in their C-termini. By contrast, the NF-κB1 and NF-κB2 proteins are synthesized as large precursors, p105, and p100, which undergo processing to generate the mature NF-κB subunits, p50 and p52, respectively, which processing is mediated by the ubiquitin/proteasome pathway and involves selective degradation of their C-terminal region containing ankyrin repeats.

NF-κB is important in regulating cellular responses because it belongs to the category of “rapid-acting” primary transcription factors and is a first responder to harmful cellular stimuli. That is NF-κB (along with transcription factors such as c-Jun, STATs, and nuclear hormone receptors) is a transcription factor that is present in cells in an inactive state and does not require new protein synthesis in order to become activated. Known inducers of NF-κB activity are highly variable and include reactive oxygen species (ROS), tumor necrosis factor alpha (TNFα), interleukin 1-beta (IL-1β), bacterial lipopolysaccharides (LPS), isoproterenol, cocaine, and ionizing radiation.

Receptor activator of NF-κB (RANK), which is a type of tumor necrosis factor receptor (TNFR), is a central activator of NF-κB. Osteoprotegerin (OPG), which is a decoy receptor homolog for RANK ligand (RANKL), inhibits RANK by binding to RANKL, and, thus, osteoprotegerin is tightly involved in regulating NF-κB activation. In unstimulated cells, the NF-κB dimers are sequestered in the cytoplasm by a family of inhibitors, called IκBs (inhibitor of κB), which are proteins that contain multiple copies of a sequence called ankyrin repeats. By virtue of their ankyrin repeat domains, the IκB proteins mask the nuclear localization signals (NLS) of NF-κB proteins and keep them sequestered in an inactive state in the cytoplasm.

NF-κB is widely used by eukaryotic cells as a regulator of genes that control cell proliferation and cell survival. As such, many different types of human tumors have aberrantly regulated NF-κB, i.e., NF-κB is constitutively active. Active NF-κB turns on the expression of genes that keep the cell proliferating and protect the cell from conditions that would otherwise cause it to die via apoptosis. Normal cells can die when removed from the tissue they belong to, or when their genome cannot operate in harmony with tissue function. Each of these events depend on feedback regulation of NF-κB, which fails in cancer. Additionally, because NF-κB controls many genes involved in inflammation, it is not surprising that NF-κB is constitutively active in many inflammatory diseases, such as inflammatory bowel disease, arthritis, sepsis, gastritis, asthma, and atherosclerosis, among others.

### The Cancer Genome Atlas (TCGA)

The Cancer Genome Atlas (TCGA) is a project to catalogue genetic mutations responsible for cancer, using genome sequencing and bioinformatics (Cancer Genome Atlas N. Genomic Classification of Cutaneous Melanoma. 2015 Cell, 161(7):1681-96, incorporated herein by reference). TCGA applies high-throughput genome analysis techniques to improve the ability to diagnose, treat, and prevent cancer through a better understanding of the genetic basis of this disease.

The project scheduled 500 patient samples, more than most genomics studies, and used different techniques to analyze the patient samples. Techniques include gene expression profiling, copy number variation profiling, SNP genotyping, genome wide DNA methylation profiling, microRNA profiling, and exon sequencing of at least 1,200 genes. TCGA is sequencing the entire genomes of some tumors, including at least 6,000 candidate genes and microRNA sequences. This targeted sequencing is being performed by all three sequencing centers using hybrid-capture technology. In phase II, TCGA is performing whole exon sequencing on 80% of the cases and whole genome sequencing on 80% of the cases used in the project.

### Gene Expression Profiling

In general, methods of gene expression profiling can be divided into two large groups: methods based on hybridization analysis of polynucleotides, and methods based on sequencing of polynucleotides. Methods known in the art for the quantification of mRNA expression in a sample include northern blotting and in situ hybridization, RNAse protection assays, RNA-seq, and reverse transcription polymerase chain reaction (RT-PCR). Alternatively, antibodies are employed that recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). For example, RT-PCR is used to compare mRNA levels in different sample populations, in normal and tumor tissues, with or without drug treatment, to characterize patterns of gene expression, to discriminate between closely related mRNAs, and/or to analyze RNA structure.

In some cases, a first step in gene expression profiling by RT-PCR is the reverse transcription of the RNA template into cDNA, followed by amplification in a PCR reaction. For example, extracted RNA is reverse-transcribed using a GeneAmp RNA PCR kit (Perkin Elmer, Calif., USA), following the manufacturer's instructions. The cDNA is then used as template in a subsequent PCR amplification and quantitative analysis using, for example, a TaqMan RTM (Life Technologies, Inc., Grand Island, N.Y.) assay.

### Microarrays

Differential gene expression can also be identified, or confirmed using a microarray technique. In these methods, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines and corresponding normal tissues or cell lines. Thus, RNA is isolated from a variety of primary tumors or tumor cell lines. If the source of mRNA is a primary tumor, mRNA is extracted from frozen or archived tissue samples.

In the microarray technique, PCR-amplified inserts of cDNA clones are applied to a substrate in a dense array. The microarrayed genes, immobilized on the microchip, are suitable for hybridization under stringent conditions.

In some cases, fluorescently labeled cDNA probes are generated through incorporation of fluorescent nucleotides by reverse transcription of RNA extracted from tissues of interest (e.g., melanoma tissue). Labeled cDNA probes applied to the chip hybridize with specificity to loci of DNA on the array. After washing to remove non-specifically bound probes, the chip is scanned by confocal laser microscopy or by another detection method, such as a charge-coupled device (CCD) camera. Quantification of hybridization of each arrayed element allows for assessment of corresponding mRNA abundance.

In some configurations, dual color fluorescence is used. With dual color fluorescence, separately labeled cDNA probes generated from two sources of RNA are hybridized pairwise to the array. The relative abundance of the transcripts from the two sources corresponding to each specified gene is thus determined simultaneously. In various configurations, the miniaturized scale of the hybridization can afford a convenient and rapid evaluation of the expression pattern for large numbers of genes. In various configurations, such methods can have sensitivity required to detect rare transcripts, which are expressed at fewer than 1000, fewer than 100, or fewer than 10 copies per cell. In various configurations, such methods can detect at least approximately two-fold differences in expression levels (Schena et al., Proc. Natl. Acad. Sci. USA 93(2): 106-149 (1996)). In various configurations, microarray analysis is performed by commercially available equipment, following manufacturer's protocols, such as by using the Affymetrix GenChip technology, or Incyte's microarray technology.

### RNA-Seq

RNA sequencing (RNA-seq), also called whole transcriptome shotgun sequencing (WTSS), uses next-generation sequencing (NGS) to reveal the presence and quantity of RNA in a biological sample at a given moment in time.

RNA-Seq is used to analyze the continually changing cellular transcriptome. See, e.g., Wang et al., 2009 Nat Rev Genet, 10(1): 57-63, incorporated herein by reference. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

Prior to RNA-Seq, gene expression studies were done with hybridization-based microarrays. Issues with microarrays include cross-hybridization artifacts, poor quantification of lowly and highly expressed genes, and needing to know the sequence of interest. Because of these technical issues, transcriptomics transitioned to sequencing-based methods. These progressed from Sanger sequencing of Expressed Sequence Tag libraries, to chemical tag-based methods (e.g., serial analysis of gene expression), and finally to the current technology, NGS of cDNA (notably RNA-Seq).

An exemplary human NF-κB amino acid sequence is set forth below (SEQ ID NO: 2; GenBank Accession No: CAA43716, Version 1, incorporated herein by reference):

| | |
|---|---|
| 1 | mescynpgld giieyddfkl nssivepkep apetadgpyl viveqpkqrg frfrygcegp |
| 61 | shgglpgass ekgrktyptv kicnyegpak ievdlvthsd pprahahslv gkqcselgic |
| 121 | aysvgpkdmt aqfnnlgvlh vtkknmmgtm iqklqrqrlr srpqglteae qreleqeake |
| 181 | lkkvmdlsiv rlrfsaflra sdgsfslplk pvtsqpihds kspgasnlki srmdktagsv |
| 241 | rggdevyllc dkvqkddiev rfyeddengw qafgdfsptd vhkqyaivfr tppyhkmkie |
| 301 | rpvtvflqlk rkrggdvsds kqftyyplve dkeevqrkrr kalptfsqpf gggshmgggs |
| 361 | ggaaggygga gggegvlmeg gvkvreavee knlgeagrgl hacnpafgrp rqavt |

An exemplary human NF-κB nucleic acid sequence is set forth below (SEQ ID NO: 3; GenBank Accession No: X61498, Version 1, incorporated herein by reference):

| | |
|---|---|
| 1 | actttcctgc cccttccccg gccaagccca actccggatc tcgctctcca ccggatctca |
| 61 | cccgccacac ccggacaggc ggctggagga ggcgggcgtc taaaattctg ggaagcagaa |
| 121 | cctggccgga gccactagac agagccgggc ctagcccaga gacatggaga gttgctacaa |
| 181 | cccaggtctg gatggtatta ttgaatatga tgatttcaaa ttgaactcct ccattgtgga |
| 241 | acccaaggag ccagccccag aaacagctga tggcccctac ctggtgatcg tggaacagcc |
| 301 | taagcagaga ggcttccgat ttcgatatgg ctgtgaaggc ccctcccatg gaggactgcc |
| 361 | cggtgcctcc agtgagaagg gccgaaagac ctatcccact gtcaagatct gtaactacga |
| 421 | gggaccagcc aagatcgagg tggacctggt aacacacagt gacccacctc gtgctcatgc |
| 481 | ccacagtctg gtgggcaagc aatgctcgga gctggggatc tgcgccgttt ctgtggggcc |
| 541 | caaggacatg actgcccaat ttaacaacct gggtgtcctg catgtgacta agaagaacat |
| 601 | gatggggact atgatacaaa aacttcagag gcagcggctc cgctctaggc cccagggcct |
| 661 | tacggaggcc gagcagcggg agctggagca agaggccaaa gaactgaaga aggtgatgga |
| 721 | tctgagtata gtgcggctgc gcttctctgc cttccttaga gccagtgatg gctccttctc |
| 781 | cctgcccctg aagccagtca cctcccagcc catccatgat agcaaatctc cgggggcatc |
| 841 | aaacctgaag atttctcgaa tggacaagac agcaggctct gtgcggggtg gagatgaagt |
| 901 | ttatctgctt tgtgacaagg tgcagaaaga tgacattgag gttcggttct atgaggatga |
| 961 | tgagaatgga tggcaggcct ttggggactt ctctcccaca gatgtgcata aacagtatgc |
| 1021 | cattgtgttc cggacacccc cctatcacaa gatgaagatt gagcggcctg taacagtgtt |
| 1081 | tctgcaactg aaacgcaagc gaggagggga cgtgtctgat tccaaacagt tcacctatta |
| 1141 | ccctctggtg gaagacaagg aagaggtgca gcggaagcgg aggaaggcct tgcccacctt |
| 1201 | ctcccagccc ttcgggggtg gctcccacat gggtggaggc tctgggggtg cagccggggg |
| 1261 | ctacggagga gctggaggag gtggcagcct cggtttcttc ccctcctccc tggcctacag |
| 1321 | cccctaccag tccggcgcgg gccccatgcg gtgctacccg ggaggcgggg gcggggcgca |
| 1381 | gatggccgcc acggtgccca gcagggactc cggggaggaa gccgcggagc cgagcgcccc |
| 1441 | ctccaggacc ccccagtgcg agccgcaggc cccggagatg ctgcagcgag ctcgagagta |
| 1501 | caacgcgcgc ctgttcggcc tggcgcacgc agccccgagc cctactcgac tactgcgtca |
| 1561 | ccgcggacgc cgcgcgctgc tggcgggaca gcgccacctg ctgacggcgc aggacgagaa |
| 1621 | cggagacaca ccactgcacc tagccatcat ccacgggcag accagtgtca ttgagcagat |
| 1681 | agtctatgtc atccaccacg cccaggacct cggcgttgtc aacctcacca accacctgca |
| 1741 | ccagacgccc ctgcacctgg cggtgatcac ggggcagacg agtgtggtga gctttctgct |
| 1801 | gcgggtaggt gcagacccag ctctgctgga tcggcatgga gactcagcca tgcatctggc |
| 1861 | gctgcgggca ggcgctggtg ctcctgagct gctgcgtgca ctgcttcaga gtggagctcc |
| 1921 | tgctgtgccc cagctgttgc atatgcctga ctttgaggga ctgtatccag tacacctggc |
| 1981 | ggtccgagcc cgaagccctg agtgcctgga tctgctggtg gacagtgggg ctgaagtgga |
| 2041 | ggccacagag cggcaggggg gacgaacagc cttgcatcta gccacagaga tggaggagct |
| 2101 | ggggttggtc acccatctgg tcaccaagct ccgggccaac gtgaacgctc gcacctttgc |
| 2161 | gggaaacaca cccctgcacc tggcagctgg actggggtac ccgaccctca cccgcctcct |
| 2221 | tctgaaggct ggtgctgaca tccatgctga aaacgaggag cccctgtgcc cactgccttc |
| 2281 | accccctacc tctgatagcg actcggactc tgaagggcct gagaaggaca cccgaagcag |
| 2341 | cttccggggc cacacgcctc ttgacctcac ttgcagcacc ttggtgaaga ccttgctgct |
| 2401 | aaatgctgct cagaacacca tggagccacc cctgaccccg cccagcccag cagggccggg |
| 2461 | actgtcactt ggtgatacag ctctgcagaa cctggagcag ctgctagacg ggccagaagc |
| 2521 | ccagggcagc tgggcagagc tggcagagcg tctggggctg cgcagcctgg tagacacgta |
| 2581 | ccgacagaca acctcaccca gtggcagcct cctgcgcagc tacgagctgg ctggcgggga |
| 2641 | cctggcaggt ctactggagg ccctgtctga catgggccta gaggagggag tgaggctgct |
| 2701 | gaggggtcca gaaacccgag acaagctgcc cagcacagag gtgaaggaag acagtgcgta |
| 2761 | cgggagccag tcagtggagc aggaggcaga gaagctgggc ccaccccctg agccaccagg |
| 2821 | agggctctcg cacgggcacc cccagcctca ggtgactgac ctgctgcctg cccccagccc |
| 2881 | ccttcccgga ccccctgtac agcgtcccca cctatttcaa atcttattta acaccccaca |
| 2941 | cccacccctc agttgggaca aataaaggat tctcatggga aggggaggac cccgaattcc |
| 3001 | t |

An exemplary human BIRC3 amino acid sequence is set forth below (SEQ ID NO: 4; GenBank Accession No: NP_892007, Version NP_892007.1, incorporated herein by reference):

| | |
|---|---|
| 1 | mnivensifl snlmksantf elkydlscel yrmstystfp agvpvsersl aragfyytgv |
| 61 | ndkvkcfccg lmldnwkrgd sptekhkkly pscrfvqsln svnnleatsq ptfpssvtns |
| 121 | thsllpgten sgyfrgsysn spsnpvnsra nqdfsalmrs syhcamnnen arlltfqtwp |
| 181 | ltflsptdla kagfyyigpg drvacfacgg klsnwepkdn amsehlrhfp kcpfienqlq |
| 241 | dtsrytvsnl smqthaarfk tffnwpssvl vnpeqlasag fyyvgnsddv kcfccdgglr |
| 301 | cwesgddpwv qhakwfprce ylirikgqef irqvqasyph lleqllstsd spgdenaess |
| 361 | iihfepgedh sedaimmntp vinaavemgf srslvkqtvq rkilatgeny rlvndlvldl |
| 421 | lnaedeiree ererateeke sndlllirkn rmalfqhltc vipildsllt agiineqehd |
| 481 | vikqktqtsl qarelidtil vkgniaatvf rnslqeaeav lyehlfvqqd ikyiptedvs |
| 541 | dlpveeqlrr lqeertckvc mdkevsivfi pcghlvvckd capslrkcpi crstikgtvr |
| 601 | tfls |

An exemplary human BIRC3 nucleic acid sequence is set forth below (SEQ ID NO: 5; GenBank Accession No: NM_001165 XM_005271534, Version NM_001165.4, incorporated herein by reference):

| | |
|---|---|
| 1 | gcatttaaaa gacagcgtga gactcgcgcc ctccggcacg gaaaaggcca ggcgacaggt |
| 61 | gtcgcttgaa aagactgggc ttgtccttgc tggtgcatgc gtcgtcggcc tctgggcagc |
| 121 | aggtttacaa aggaggaaaa cgacttcttc tagatttttt tttcagtttc ttctataaat |
| 181 | caaaacatct caaaatggag acctaaaatc cttaaaggga cttagtctaa tctcgggagg |
| 241 | tagttttgtg catgggtaaa caaattaagt attaactggt gttttactat ccaaagaatg |
| 301 | ctaattttat aaacatgatc gagttatata aggtatacca taatgagttt gattttgaat |
| 361 | ttgatttgtg gaaataaagg aaaagtgatt ctagctgggg catattgtta aagcattttt |
| 421 | ttcagagttg gccaggcagt ctcctactgg cacattctcc cattatgtag aatagaaata |
| 481 | gtacctgtgt ttgggaaaga ttttaaaatg agtgacagtt atttggaaca aagagctaat |
| 541 | aatcaatcca ctgcaaatta aagaaacatg cagatgaaag ttttgacaca ttaaaatact |
| 601 | tctacagtga caaagaaaaa tcaagaacaa agctttttga tatgtgcaac aaatttagag |
| 661 | gaagtaaaaa gataaatgtg atgattggtc aagaaattat ccagttattt acaaggccac |
| 721 | tgatatttta aacgtccaaa agtttgttta aatgggctgt taccgctgag aatgatgagg |
| 781 | atgagaatga tggttgaagg ttacatttta ggaaatgaag aaacttagaa aattaatata |
| 841 | aagacagtga tgaatacaaa gaagattttt ataacaatgt gtaaaatttt tggccaggga |
| 901 | aaggaatatt gaagttagat acaattactt acctttgagg gaaataattg ttggtaatga |
| 961 | gatgtgatgt ttctcctgcc acctggaaac aaagcattga agtctgcagt tgaaaagccc |
| 1021 | aacgtctgtg agatccagga aaccatgctt gcaaaccact ggtaaaaaaa aaaaaaaaaa |
| 1081 | aaaaaaaaag ccacagtgac ttgcttattg gtcattgcta gtattatcga ctcagaacct |
| 1141 | ctttactaat ggctagtaaa tcataattga gaaattctga attttgacaa ggtctctgct |
| 1201 | gttgaaatgg taaatttatt attttttttg tcatgataaa ttctggttca aggtatgcta |
| 1261 | tccatgaaat aatttctgac caaaactaaa ttgatgcaat ttgattatcc atcttagcct |
| 1321 | acagatggca tctggtaact tttgactgtt ttaaaaaata aatccactat cagagtagat |
| 1381 | ttgatgttgg cttcagaaac atttagaaaa acaaaagttc aaaaatgttt tcaggaggtg |
| 1441 | ataagttgaa taactctaca atgttagttc tttgaggggg acaaaaaatt taaaatcttt |
| 1501 | gaaaggtctt attttacagc catatctaaa ttatcttaag aaaattttta acaaagggaa |
| 1561 | tgaaatatat atcatgattc tgtttttcca aaagtaacct gaatatagca atgaagttca |
| 1621 | gttttgttat tggtagtttg ggcagagtct ctttttgcag cacctgttgt ctaccataat |
| 1681 | tacagaggac atttccatgt tctagccaag tatactatta gaataaaaaa acttaacatt |
| 1741 | gagttgcttc aacagcatga aactgagtcc aaaagaccaa atgaacaaac acattaatct |
| 1801 | ctgattattt attttaaata gaatatttaa ttgtgtaaga tctaatagta tcattatact |
| 1861 | taagcaatca tattcctgat gatctatggg aaataactat tatttaatta atattgaaac |
| 1921 | caggttttaa gatgtgttag ccagtcctgt tactagtaaa tctctttatt tggagagaaa |
| 1981 | ttttagattg ttttgttctc cttattagaa ggattgtaga aagaaaaaaa tgactaattg |
| 2041 | gagaaaaatt ggggatatat catatttcac tgaattcaaa atgtcttcag ttgtaaatct |
| 2101 | taccattatt ttacgtacct ctaagaaata aaagtgcttc taattaaaat atgatgtcat |
| 2161 | taattatgaa atacttcttg ataacagaag ttttaaaata gccatcttag aatcagtgaa |
| 2221 | atatggtaat gtattatttt cctcctttga gttaggtctt gtgctttttt ttcctggcca |
| 2281 | ctaaatttca caatttccaa aaagcaaaat aaacatattc tgaatatttt tgctgtgaaa |
| 2341 | cacttgacag cagagctttc caccatgaaa agaagcttca tgagtcacac attacatctt |
| 2401 | tgggttgatt gaatgccact gaaacattct agtagcctgg agaagttgac ctacctgtgg |
| 2461 | agatgcctgc cattaaatgg catcctgatg gcttaataca catcactctt ctgtgaaggg |
| 2521 | ttttaatttt caacacagct tactctgtag catcatgttt acattgtatg tataaagatt |
| 2581 | atacaaaggt gcaattgtgt atttcttcct taaaatgtat cagtatagga tttagaatct |
| 2641 | ccatgttgaa actctaaatg catagaaata aaaataataa aaaatttttc attttggctt |
| 2701 | ttcagcctag tattaaaact gataaaagca aagccatgca caaaactacc tccctagaga |
| 2761 | aaggctagtc ccttttcttc cccattcatt tcattatgaa catagtagaa aacagcatat |
| 2821 | tcttatcaaa tttgatgaaa agcgccaaca cgtttgaact gaaatacgac ttgtcatgtg |
| 2881 | aactgtaccg aatgtctacg tattccactt ttcctgctgg ggttcctgtc tcagaaagga |
| 2941 | gtcttgctcg tgctggtttc tattacactg gtgtgaatga caaggtcaaa tgcttctgtt |
| 3001 | gtggcctgat gctggataac tggaaaagag gagacagtcc tactgaaaag cataaaaagt |
| 3061 | tgtatcctag ctgcagattc gttcagagtc taaattccgt taacaacttg gaagctacct |
| 3121 | ctcagcctac ttttccttct tcagtaacaa attccacaca ctcattactt ccgggtacag |
| 3181 | aaaacagtgg atatttccgt ggctcttatt caaactctcc atcaaatcct gtaaactcca |
| 3241 | gagcaaatca agatttttct gccttgatga gaagttccta ccactgtgca atgaataacg |
| 3301 | aaaatgccag attacttact tttcagacat ggccattgac ttttctgtcg ccaacagatc |
| 3361 | tggcaaaagc aggcttttac tacataggac ctggagacag agtggcttgc tttgcctgtg |
| 3421 | gtggaaaatt gagcaattgg gaaccgaagg ataatgctat gtcagaacac ctgagacatt |
| 3481 | ttcccaaatg cccatttata gaaaatcagc ttcaagacac ttcaagatac acagtttcta |
| 3541 | atctgagcat gcagacacat gcagcccgct ttaaaacatt ctttaactgg ccctctagtg |
| 3601 | ttctagttaa tcctgagcag cttgcaagtg cgggttttta ttatgtgggt aacagtgatg |
| 3661 | atgtcaaatg cttttgctgt gatggtggac tcaggtgttg ggaatctgga gatgatccat |
| 3721 | gggttcaaca tgccaagtgg tttccaaggt gtgagtactt gataagaatt aaaggacagg |
| 3781 | agttcatccg tcaagttcaa gccagttacc ctcatctact tgaacagctg ctatccacat |
| 3841 | cagacagccc aggagatgaa aatgcagagt catcaattat ccattttgaa cctggagaag |
| 3901 | accattcaga agatgcaatc atgatgaata ctcctgtgat taatgctgcc gtggaaatgg |
| 3961 | gctttagtag aagcctggta aaacagacag ttcagagaaa aatcctagca actggagaga |
| 4021 | attatagact agtcaatgat cttgtgttag acttactcaa tgcagaagat gaaataaggg |
| 4081 | aagaggagag agaaagagca actgaggaaa aagaatcaaa tgatttatta ttaatccgga |
| 4141 | agaatagaat ggcacttttt caacatttga cttgtgtaat tccaatcctg gatagtctac |
| 4201 | taactgccgg aattattaat gaacaagaac atgatgttat taaacagaag acacagacgt |
| 4261 | ctttacaagc aagagaactg attgatacga ttttagtaaa aggaaatatt gcagccactg |
| 4321 | tattcagaaa ctctctgcaa gaagctgaag ctgtgttata tgagcattta tttgtgcaac |
| 4381 | aggacataaa atatattccc acagaagatg tttcagatct accagtggaa gaacaattgc |
| 4441 | ggagactaca agaagaaaga acatgtaaag tgtgtatgga caaagaagtg tccatagtgt |
| 4501 | ttattccttg tggtcatcta gtagtatgca aagattgtgc tccttcttta agaaagtgtc |
| 4561 | ctatttgtag gagtacaatc aagggtacag ttcgtacatt tctttcatga agaagaacca |
| 4621 | aaacatcgtc taaactttag aattaattta ttaaatgtat tataacttta acttttatcc |
| 4681 | taatttggtt tccttaaaat ttttatttat ttacaactca aaaaacattg ttttgtgtaa |
| 4741 | catatttata tatgtatcta aaccatatga acatatattt tttagaaact aagagaatga |
| 4801 | taggcttttg ttcttatgaa cgaaaaagag gtagcactac aaacacaata ttcaatcaaa |
| 4861 | atttcagcat tattgaaatt gtaagtgaag taaaacttaa gatatttgag ttaaccttta |
| 4921 | agaattttaa atattttggc attgtactaa taccgggaac atgaagccag gtgtggtggt |
| 4981 | atgtgcctgt agtcccaggc tgaggcaaga gaattacttg agcccaggag tttgaatcca |
| 5041 | tcctgggcag catactgaga ccctgccttt aaaaacaaac agaacaaaaa caaaacacca |
| 5101 | gggacacatt tctctgtctt ttttgatcag tgtcctatac atcgaaggtg tgcatatatg |
| 5161 | ttgaatgaca ttttagggac atggtgtttt tataaagaat tctgtgagaa aaaatttaat |
| 5221 | aaagcaacaa aaattactct tattcttcat tgctttattt caatgacatt ggatagttta |
| 5281 | gtcactccca gactctttcc ataccttctt aaagcctctc aaatattgaa ctacagttta |
| 5341 | tactccttcc cataagatgc ttcttcattg acacttgtag aacacggggt caacacatca |
| 5401 | taaaatctat tatggaatgc ctgagacaag aatcaaacag tccctttagt aagtttgttt |
| 5461 | attcacttct ctattgattc attcaagaag tctcatgcca gccccaccta ttggaagaag |
| 5521 | gtctgagttt tattcttatc tctttggtat taattctgaa acttagaaag tacactggtt |
| 5581 | agcaatgctt gggaccaaca ggttgttctg gtaaataaat ctgtttcata ttgtcagtgc |
| 5641 | aacaaaatgt ccccctctgc attatgttat tggtactcaa cacgtccgag tcataactct |
| 5701 | gtcctttgct tcttatagag gtattaggtc ttcaagagca gaagtaagac tgtaataggg |
| 5761 | aatactcagg ggaaggcagg caaaggctag tcatctaaac cagttctaga tgtctgtata |
| 5821 | ggggcagatg gctctgtaag ggcagaaggg aaagacccct tcataagggt cacagctgac |
| 5881 | aatcctataa caaaagacag gttaacaaga gaaaaactta acaaatttat ttaatcacag |
| 5941 | atttacatca ccggggagcc ttcgtaatga agatccaaaa ttacagggga aactgtgcat |
| 6001 | ttttatgctt aggtttgata atgaatggac agccctgaag aatagtgatt ggaaaaaaag |
| 6061 | gatatgatct aatgggaata gacacaggtt ggggacccag caaggcctgt ctgttcagat |
| 6121 | tattcttggt ctctgtgcag cattccttcc tcctggatat agggcagggc ctgtatggga |
| 6181 | tggggatatt ataacctgct atcaagcaag gtaggtcaga gaatttattt atggccagct |
| 6241 | cttacatagt taggtgagga aagattagag tactatcttt aagatgtaag tctggcattg |
| 6301 | tggaaagatg gttccagttt ctatgaccta ccttggggaa gaggaattca agtttctgtg |
| 6361 | gcttgccttc agggagaatg aggctgagac aggagggcag gataacatca gagaaaaact |
| 6421 | ttgcttctga ggccttcact ttgggttttc tgagccccaa catctgctag tgttgtaaag |
| 6481 | agaacaatta gggaccaagt gaggggagga aagaatccat ctctgcattc tgatgctggg |
| 6541 | agacttattt ccttgaaatg caattgattt tgcctctgct aagaggctct gctggctacc |
| 6601 | catgtactag ccagtgtcct gcatgggtgc taggctgaat tatttgtaat tgtgcttagg |
| 6661 | tgatttgtaa ctcaggtata gggtatttaa atagtaggca ccctttttgc accatgtgtt |
| 6721 | ttttttttta tctagttctt gtatactaca gataatattt gaactttgtc atctcactgt |
| 6781 | aaaacttttg ttcatttctc attatggtaa taaatagcta ttataaccaa cccatttatt |
| 6841 | caaatatgtt atttccctaa gtgttatttt gacattttgt tttggaaaaa ataaatcacc |
| 6901 | atagataata aaaaaaaaaa aaaaaaaaaa aa |

An exemplary human IL-8 amino acid sequence is set forth below (SEQ ID NO: 6; GenBank Accession No: AAH13615, Version AAH13615.1, incorporated herein by reference):

| | |
|---|---|
| 1 | mtsklavall aaflisaalc egavlprsak elrcqcikty skpfhpkfik elrviesgph |
| 61 | canteiivkl sdgrelcldp kenwvqrvve kflkraens |

An exemplary human IL-8 nucleic acid sequence is set forth below (SEQ ID NO: 7; GenBank Accession No: BC013615, Version BC013615.1, incorporated herein by reference):

| | |
|---|---|
| 1 | gaagaaacca ccggaaggaa ccatctcact gtgtgtaaac atgacttcca agctggccgt |
| 61 | ggctctcttg gcagccttcc tgatttctgc agctctgtgt gaaggtgcag ttttgccaag |
| 121 | gagtgctaaa gaacttagat gtcagtgcat aaagacatac tccaaacctt tccaccccaa |
| 181 | atttatcaaa gaactgagag tgattgagag tggaccacac tgcgccaaca cagaaattat |
| 241 | tgtaaagctt tctgatggaa gagagctctg tctggacccc aaggaaaact gggtgcagag |
| 301 | ggttgtggag aagtttttga agagggctga gaattcataa aaaaattcat tctctgtggt |
| 361 | atccaagaat cagtgaagat gccagtgaaa cttcaagcaa atctacttca acacttcatg |
| 421 | tattgtgtgg gtctgttgta gggttgccag atgcaataca agattcctgg ttaaatttga |
| 481 | atttcagtaa acaatgaata gtttttcatt gtaccatgaa atatccagaa catacttata |
| 541 | tgtaaagtat tatttatttg aatctacaaa aaacaacaaa taatttttaa atataaggat |
| 601 | tttcctagat attgcacggg agaatataca aatagcaaaa ttgaggccaa gggccaagag |
| 661 | aatatccgaa ctttaatttc aggaattgaa tgggtttgct agaatgtgat atttgaagca |
| 721 | tcacataaaa atgatgggac aataaatttt gccataaagt caaatttagc tggaaatcct |
| 781 | ggattttttt ctgttaaatc tggcaaccct agtctgctag ccaggatcca caagtccttg |
| 841 | ttccactgtg ccttggtttc tcctttattt ctaagtggaa aaagtattag ccaccatctt |
| 901 | acctcacagt gatgttgtga ggacatgtgg aagcacttta agttttttca tcataacata |
| 961 | aattattttc aagtgtaact tattaaccta tttattattt atgtatttat ttaagcatca |
| 1021 | aatatttgtg caagaatttg gaaaaataga agatgaatca ttgattgaat agttataaag |
| 1081 | atgttatagt aaatttattt tattttagat attaaatgat gttttattag ataaatttca |
| 1141 | atcagggttt ttagattaaa caaacaaaca attgggtacc cagttaaatt ttcatttcag |
| 1201 | ataaacaaca aataattttt tagtataagt acattattgt ttatctgaaa ttttaattga |
| 1261 | actaacaatc ctagtttgat actcccagtc ttgtcattgc cagctgtgtt ggtagtgctg |
| 1321 | tgttgaatta cggaataatg agttagaact attaaaacag ccaaaactcc acagtcaata |
| 1381 | ttagtaattt cttgctggtt gaaacttgtt tattatgtac aaatagattc ttataatatt |
| 1441 | atttaaatga ctgcattttt aaatacaagg ctttatattt ttaactttaa gatgttttta |
| 1501 | tgtgctctcc aaattttttt tactgtttct gattgtatgg aaatataaaa gtaaatatga |
| 1561 | aacatttaaa atataatttg ttgtcaaagt aaaaaaaaaa aaaaa |

An exemplary human TNFAIP3 amino acid sequence is set forth below (SEQ ID NO: 8; GenBank Accession No: NP_001257437, Version NP_001257437.1, incorporated herein by reference). UniProtKB: P21580 also provides an exemplary TNFAIP3 amino acid sequence.

| | |
|---|---|
| 1 | maeqvlpqal ylsnmrkavk irertpedif kptngiihhf ktmhrytlem frtcqfcpqf |
| 61 | reiihkalid rniqatlesq kklnwcrevr klvalktngd gnclmhatsq ymwgvqdtdl |
| 121 | vlrkalfstl ketdtrnfkf rwqleslksq efvetglcyd trnwndewdn likmastdtp |
| 181 | marsglqyns leeihifvlc nilrrpiivi sdkmlrsles gsnfaplkvg giylplhwpa |
| 241 | qecyrypivl gydshhfvpl vtlkdsgpei ravplvnrdr grfedlkvhf ltdpenemke |
| 301 | kllkeylmvi eipvqgwdhg tthlinaakl deanlpkein lvddyfelvq heykkwqens |
| 361 | eqgrreghaq npmepsvpql slmdvkcetp ncpffmsvnt gplchecser rqknqnklpk |
| 421 | lnskpgpegl pgmalgasrg eayeplawnp eestggphsa pptapspflf settamkcrs |
| 481 | pgcpftlnvq hngfcerchn arqlhashap dhtrhldpgk cqaclqdvtr tfngicstcf |
| 541 | krttaeasss lstslppsch qrsksdpsrl vrspsphsch ragndapagc lsqaartpgd |
| 601 | rtgtskcrka gcvyfgtpen kgfctlcfie yrenkhfaaa sgkvsptasr fqntipclgr |
| 661 | ecgtlgstmf egycqkcfie aqnqrfheak rteeqlrssq rrdvprttqs tsrpkcaras |
| 721 | cknilacrse elcmecqhpn qrmgpgahrg epapedppkq rcrapacdhf gnakcngycn |
| 781 | ecfqfkqmyg |

An exemplary human TNFAIP3 nucleic acid sequence is set forth below (SEQ ID NO: 9; GenBank Accession No: NM_001270508, Version NM_001270508.1, incorporated herein by reference):

| | |
|---|---|
| 1 | ctttggaaag tcccgtggaa atccccgggc ctacaacccg catacaactg aaacggggca |
| 61 | aagcagactg cgcagtctgc agtcttcgtg gcgggccaag cgagcttgga gcccgcgggg |
| 121 | gcggagcggt gagagcggcc gccaagagag atcacacccc cagccgaccc tgccagcgag |
| 181 | cgagcccgac cccaggcgtc catggagcgt cgcctccgcc cggtccctgc cccgaccccc |
| 241 | gcctgcggcg cgctcctgcc ttgaccagga cttgggactt tgcgaaagga tcgcggggcc |
| 301 | cggagaggta accgccgcgc ctcccggaga ggtaaccgcc gcgcctcccg gagaggtgtt |
| 361 | ggagagcaca atggctgaac aagtccttcc tcaggctttg tatttgagca atatgcggaa |
| 421 | agctgtgaag atacgggaga gaactccaga agacattttt aaacctacta atgggatcat |
| 481 | tcatcatttt aaaaccatgc accgatacac actggaaatg ttcagaactt gccagttttg |
| 541 | tcctcagttt cgggagatca tccacaaagc cctcatcgac agaaacatcc aggccaccct |
| 601 | ggaaagccag aagaaactca actggtgtcg agaagtccgg aagcttgtgg cgctgaaaac |
| 661 | gaacggtgac ggcaattgcc tcatgcatgc cacttctcag tacatgtggg gcgttcagga |
| 721 | cacagacttg gtactgagga aggcgctgtt cagcacgctc aaggaaacag acacacgcaa |
| 781 | ctttaaattc cgctggcaac tggagtctct caaatctcag gaatttgttg aaacggggct |
| 841 | ttgctatgat actcggaact ggaatgatga atgggacaat cttatcaaaa tggcttccac |
| 901 | agacacaccc atggcccgaa gtggacttca gtacaactca ctggaagaaa tacacatatt |
| 961 | tgtcctttgc aacatcctca gaaggccaat cattgtcatt tcagacaaaa tgctaagaag |
| 1021 | tttggaatca ggttccaatt tcgccccttt gaaagtgggt ggaatttact tgcctctcca |
| 1081 | ctggcctgcc caggaatgct acagataccc cattgttctc ggctatgaca gccatcattt |
| 1141 | tgtacccttg gtgaccctga aggacagtgg gcctgaaatc cgagctgttc cacttgttaa |
| 1201 | cagagaccgg ggaagatttg aagacttaaa agttcacttt ttgacagatc ctgaaaatga |
| 1261 | gatgaaggag aagctcttaa aagagtactt aatggtgata gaaatccccg tccaaggctg |
| 1321 | ggaccatggc acaactcatc tcatcaatgc cgcaaagttg gatgaagcta acttaccaaa |
| 1381 | agaaatcaat ctggtagatg attactttga acttgttcag catgagtaca agaaatggca |
| 1441 | ggaaaacagc gagcagggga ggagagaggg gcacgcccag aatcccatgg aaccttccgt |
| 1501 | gccccagctt tctctcatgg atgtaaaatg tgaaacgccc aactgcccct tcttcatgtc |
| 1561 | tgtgaacacc cagcctttat gccatgagtg ctcagagagg cggcaaaaga atcaaaacaa |
| 1621 | actcccaaag ctgaactcca agccgggccc tgaggggctc cctggcatgg cgctcggggc |
| 1681 | ctctcgggga gaagcctatg agcccttggc gtggaaccct gaggagtcca ctggggggcc |
| 1741 | tcattcggcc ccaccgacag cacccagccc ttttctgttc agtgagacca ctgccatgaa |
| 1801 | gtgcaggagc cccggctgcc ccttcacact gaatgtgcag cacaacggat tttgtgaacg |
| 1861 | ttgccacaac gcccggcaac ttcacgccag ccacgcccca gaccacacaa ggcacttgga |
| 1921 | tcccgggaag tgccaagcct gcctccagga tgttaccagg acatttaatg ggatctgcag |
| 1981 | tacttgcttc aaaaggacta cagcagaggc ctcctccagc ctcagcacca gcctccctcc |
| 2041 | ttcctgtcac cagcgttcca agtcagatcc ctcgcggctc gtccggagcc cctccccgca |
| 2101 | ttcttgccac agagctggaa acgacgcccc tgctggctgc ctgtctcaag ctgcacggac |
| 2161 | tcctggggac aggacgggga cgagcaagtg cagaaaagcc ggctgcgtgt attttgggac |
| 2221 | tccagaaaac aagggctttt gcacactgtg tttcatcgag tacagagaaa acaaacattt |
| 2281 | tgctgctgcc tcagggaaag tcagtcccac agcgtccagg ttccagaaca ccattccgtg |
| 2341 | cctggggagg gaatgcggca cccttggaag caccatgttt gaaggatact gccagaagtg |
| 2401 | tttcattgaa gctcagaatc agagatttca tgaggccaaa aggacagaag agcaactgag |
| 2461 | atcgagccag cgcagagatg tgcctcgaac cacacaaagc acctcaaggc ccaagtgcgc |
| 2521 | ccgggcctcc tgcaagaaca tcctggcctg ccgcagcgag gagctctgca tggagtgtca |
| 2581 | gcatcccaac cagaggatgg gccctggggc ccaccggggt gagcctgccc ccgaagaccc |
| 2641 | ccccaagcag cgttgccggg cccccgcctg tgatcatttt ggcaatgcca agtgcaacgg |
| 2701 | ctactgcaac gaatgctttc agttcaagca gatgtatggc taaccggaaa caggtgggtc |
| 2761 | acctcctgca agaagtgggg cctcgagctg tcagtcatca tggtgctatc ctctgaaccc |
| 2821 | ctcagctgcc actgcaacag tgggcttaag ggtgtctgag caggagagga aagataagct |
| 2881 | cttcgtggtg cccacgatgc tcaggtttgg taacccggga gtgttcccag gtggccttag |
| 2941 | aaagcaaagc ttgtaactgg caagggatga tgtcagattc agcccaaggt tcctcctctc |
| 3001 | ctaccaagca ggaggccagg aacttctttg gacttggaag gtgtgcgggg actggccgag |
| 3061 | gcccctgcac cctgcgcatc aggactgctt catcgtcttg gctgagaaag ggaaaagaca |
| 3121 | cacaagtcgc gtgggttgga gaagccagag ccattccacc tcccctcccc cagcatctct |
| 3181 | cagagatgtg aagccagatc ctcatggcag cgaggccctc tgcaagaagc tcaaggaagc |
| 3241 | tcagggaaaa tggacgtatt cagagagtgt ttgtagttca tggtttttcc ctacctgccc |
| 3301 | ggttcctttc ctgaggaccc ggcagaaatg cagaaccatc catggactgt gattctgagg |
| 3361 | ctgctgagac tgaacatgtt cacattgaca gaaaaacaag ctgctcttta taatatgcac |
| 3421 | cttttaaaaa attagaatat tttactggga agacgtgtaa ctctttgggt tattactgtc |
| 3481 | tttacttcta aagaagttag cttgaactga ggagtaaaag tgtgtacata tataatatac |
| 3541 | ccttacatta tgtatgaggg atttttttaa attatattga aatgctgccc tagaagtaca |
| 3601 | ataggaaggc taaataataa taacctgttt tctggttgtt gttggggcat gagcttgtgt |
| 3661 | atacactgct tgcataaact caaccagctg cctttttaaa gggagctcta gtcctttttg |
| 3721 | tgtaattcac tttatttatt ttattacaaa cttcaagatt atttaagtga agatatttct |
| 3781 | tcagctctgg ggaaaatgcc acagtgttct cctgagagaa catccttgct ttgagtcagg |
| 3841 | ctgtgggcaa gttcctgacc acagggagta aattggcctc tttgatacac ttttgcttgc |
| 3901 | ctccccagga aagaaggaat tgcatccaag gtatacatac atattcatcg atgtttcgtg |
| 3961 | cttctcctta tgaaactcca gctatgtaat aaaaaactat actctgtgtt ctgttaatgc |
| 4021 | ctctgagtgt cctacctcct tggagatgag atagggaagg agcagggatg agactggcaa |
| 4081 | tggtcacagg gaaagatgtg gccttttgtg atggttttat tttctgttaa cactgtgtcc |
| 4141 | tgggggggct gggaagtccc ctgcatccca tggtaccctg gtattgggac agcaaaagcc |
| 4201 | agtaaccatg agtatgagga aatctctttc tgttgctggc ttacagtttc tctgtgtgct |
| 4261 | ttgtggttgc tgtcatattt gctctagaag aaaaaaaaaa aaggagggga aatgcatttt |
| 4321 | ccccagagat aaaggctgcc attttggggg tctgtactta tggcctgaaa atatttgtga |
| 4381 | tccataactc tacacagcct ttactcatac tattaggcac actttcccct tagagccccc |
| 4441 | taagtttttc ccagacgaat ctttataatt tctttccaaa gataccaaat aaacttcagt |
| 4501 | gttttcatct aattctctta aagttgatat cttaatattt tgtgttgatc attatttcca |
| 4561 | ttcttaatgt gaaaaaaagt aattatttat acttattata aaaagtattt gaaatttgca |
| 4621 | catttaattg tccctaatag aaagccacct attctttgtt ggatttcttc aagtttttct |
| 4681 | aaataaatgt aacttttcac aagagtcaac attaaaaaat aaattattta agaacagaaa |
| 4741 | aaaaaaaaaa aaa |

### Breast Cancer

Breast cancer is a type of cancer that develops in breast tissue. Signs of breast cancer include breast lumps, breast shape change, skin dimpling, fluid coming from the nipple, or a red/scaly patch of breast skin. Bone pain, swollen lymph nodes, shortness of breath, or yellow skin may be present in those with spread of the disease beyond the breast.

Risk factors for developing breast cancer include female gender, obesity, lack of physical exercise, drinking alcohol, hormone replacement therapy during menopause, ionizing radiation, early age at first menstruation, having children late or not at all, older age, and family history. For example, in some cases, genes inherited from a person's parents, including breast cancer type 1 susceptibility protein (BRCA1) and BRCA2, among others, contribute to disease. Breast cancer most commonly develops in cells from either (1) the lining of milk ducts (ductal carcinomas); or (2) the lobules that supply the ducts with milk (lobular carcinomas); however, there are more than 18 other sub-types of breast cancer.

The diagnosis of breast cancer is confirmed by taking a biopsy of the concerning lump. Breast cancer is often treated with platinum compounds, e.g., cisplatin, carboplatin or oxaliplatin, that cause inter-strand cross-links in DNA.

### Multiple Myeloma

Multiple myeloma, also known as plasma cell myeloma, is a cancer of plasma cells, a type of white blood cell normally responsible for producing antibodies. Often, no symptoms are noticed initially; however, in advanced disease, bone pain, bleeding, frequent infections, and anemia may occur. Complications may include amyloidosis.

The cause for multiple myeloma is generally unknown. Risk factors include drinking alcohol and obesity. The underlying mechanism of disease involves abnormal plasma cells producing abnormal antibodies which can cause kidney problems and overly thick blood. Additionally, the plasma cells can also form a mass in the bone marrow or soft tissue. When only one mass is present, it is known as a “plasmacytoma.” More than one mass is known as “multiple myeloma.”

Multiple myeloma is diagnosed based on blood or urine tests finding abnormal antibodies, bone marrow biopsy finding cancerous plasma cells, and medical imaging finding bone lesions. High blood calcium levels are often associated with this disease. Multiple myeloma is considered treatable, but generally incurable. Treatment with steroids, chemotherapy, thalidomide or lenalidomide, and/or stem cell transplant can lead to remission of disease. Bisphosphonates and radiation therapy are sometimes used to reduce pain from bone lesions.

### Leukemia

Leukemia includes a group of cancers that usually begin in the bone marrow and result in high numbers of abnormal white blood cells called blasts or leukemia cells. Symptoms may include bleeding, bruising, feeling tired, fever, and an increased risk of infections. Risk factors for developing leukemia include smoking, ionizing radiation, some chemicals (e.g., benzene), prior chemotherapy, Down syndrome, and people with a family history of leukemia. There are four main types of leukemia: acute lymphoblastic leukemia (ALL), acute myeloid leukemia (AML), chronic lymphocytic leukemia (CLL) and chronic myeloid leukemia (CIVIL), as well as a number of less common types of leukemia.

Diagnosis is typically with blood tests or bone marrow biopsy. Treatment typically includes some combination of chemotherapy, radiation therapy, targeted therapy, and/or bone marrow transplant.

### Autoimmune Diseases

An autoimmune disease is a condition arising from an abnormal immune response to a normal body part. There are at least 80 types of autoimmune diseases, and nearly any body part can be involved. The causes for autoimmune diseases are generally unknown; however, some autoimmune diseases, such as lupus, run in families, and certain cases may be triggered by infections or other environmental factors. Some common diseases that are generally considered autoimmune include celiac disease, diabetes mellitus type 1, Graves' disease, inflammatory bowel disease, multiple sclerosis, psoriasis, rheumatoid arthritis, and systemic lupus erythematosus.

Treatment depends on the type and severity of the condition. Nonsteroidal anti-inflammatory drugs (NSAIDs) and immunosuppressants are often used to manage symptoms associated with the disease.

### Hyperproliferative Disorders/Neoplasias

The NF-κB inhibitors (e.g., harmine) described herein are useful to treat any hyperproliferative disorder or inflammatory disease driven by increased NF-κB activity. It is contemplated that the methods described herein are particularly useful when the individual has a hyperproliferative disorder characterized by an elevated NF-κB activity, e.g., a neoplasia.

Hyperproliferative disorders include cancerous disease states. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The compounds described herein, e.g., a NF-κB inhibitor (e.g., harmine or related compounds), can be used to treat or prevent a variety of hyperproliferative disorders. In some cases, the compounds of the invention are used to treat a cancer with elevated NF-κB activity (e.g., breast cancer and leukemia). For example, the invention is used to treat a solid tumor. In another aspect, the solid tumor is breast cancer, melanoma, colon cancer, ovarian cancer, pancreatic cancer, lung cancer, hepatic cancer, head and neck cancer, prostate cancer, and brain cancer. In another example, the hyperproliferative disorder is a hematological cancer such as leukemia or multiple myeloma. Leukemia includes acute lymphoblastic leukemia, acute myelogenous leukemia, chronic myelogenous leukemia, Hodgkin's disease, non-Hodgkin's lymphoma, T-cell lymphoma, B-cell lymphoma, and chronic lymphocytic leukemia. The described herein are also used to treat additional hyperproliferative disorders including but not limited to, cancer of the head, neck, eye, skin, mouth, throat, esophagus, chest, bone, lung, colon, sigmoid, rectum, stomach, prostate, ovary, testicle, kidney, liver, pancreas, brain, intestine, heart, or adrenals (for a review of such disorders, see Fishman et al., 1985, Medicine, 2d Ed., J.B. Lippincott Co., Philadelphia, incorporated herein by reference).

The medical practitioner can diagnose the patient using any of the conventional cancer screening methods including, but not limited to physical examination (e.g., prostate examination, breast examination, lymph nodes examination, abdominal examination, skin surveillance), visual methods (e.g., colonoscopy, bronchoscopy, endoscopy), PAP smear analyses (cervical cancer), stool guaiac analyses, blood tests (e.g., complete blood count (CBC) test), blood chemistries including liver function tests, prostate specific antigen (PSA) test, carcinoembryonic antigen (CEA) test, cancer antigen (CA)-125 test, alpha-fetoprotein (AFP)), karyotyping analyses, bone marrow analyses (e.g., in cases of hematological malignancies), histology, cytology, a sputum analysis, and imaging methods (e.g., computed tomography (CT), magnetic resonance imaging (MRI), ultrasound, X-ray imaging, mammography imaging, bone scans).

### Administration of NF-κB Inhibitors

Hyperproliferative disorders, including, but not limited to cancer, neoplasms, tumors, metastases, or any disease or disorder characterized by uncontrolled cell growth as known in the art and described herein, can be treated, suppressed, delayed, managed, inhibited or prevented by administering to a subject in need thereof a prophylactically effective regimen or a therapeutically effective regimen, the regimen comprising administering to the patient a compound of the invention, e.g., an NF-κB inhibitor. The invention as it applies to cancer encompasses the treatment, suppression, delaying, management, inhibiting of growth and/or progression, and prevention of cancer or neoplastic disease as described herein.

One aspect of the invention relates to a method of preventing, treating, and/or managing cancer in a patient (e.g., a human patient), the method comprising administering to the patient a prophylactically effective regimen or a therapeutically effective regimen, the regimen comprising administering to the patient a compound of the invention or a composition of the invention, e.g., a NF-κB inhibitor, wherein the patient has been diagnosed with cancer. The amount of a compound of the invention used in the prophylactic and/or therapeutic regimens which will be effective in the prevention, treatment, and/or management of cancer can be based on the currently prescribed dosage of the compound as well as assessed by methods disclosed herein.

In one example, the cancer is a hematologic cancer. For instance, the cancer is leukemia, lymphoma, or myeloma. In another example, the cancer is a solid tumor. In some cases, the patient has undergone a primary therapy to reduce the bulk of a solid tumor prior to therapy with the compositions and methods described herein. For example, the primary therapy to reduce the tumor bulk size is a therapy other than a compound or composition of the invention. For example, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, embryonal brain tumor, primitive neuroectodermal tumor (PNET), or choroid plexus tumor.

In one aspect, the patient has received or is receiving another therapy. In another aspect, the patient has not previously received a therapy for the prevention, treatment, and/or management of the cancer.

Another aspect of the invention relates to a method of preventing, treating, and/or managing cancer, wherein the patient received another therapy. In some embodiments, the prior therapy is, for example, chemotherapy, radioimmunotherapy, toxin therapy, prodrug-activating enzyme therapy, antibody therapy, surgical therapy, immunotherapy, radiation therapy, targeted therapy, or any combination thereof. In some embodiments, the prior therapy has failed in the patient. In some cases, the therapeutically effective regimen comprising administration of a composition of the invention is administered to the patient immediately after patient has undergone the prior therapy. For instance, in certain cases, the outcome of the prior therapy may be unknown before the patient is administered a compound of the invention.

In some cases, the therapeutic regimen results in a reduction in the cancer cell population in the patient. In one example, the patient undergoing the therapeutic regimen is monitored to determine whether the regimen has resulted in a reduction in the cancer cell population in the patient. Typically, the monitoring of the cancer cell population is conducted by detecting the number or amount of cancer cells in a specimen extracted from the patient. Methods of detecting the number or amount of cancer cells in a specimen are known in the art. This monitoring step is typically performed at least 1, 2, 4, 6, 8, 10, 12, 14, 15, 16, 18, 20, or 30 days after the patient begins receiving the regimen.

In one aspect, the specimen may be a blood specimen, wherein the number or amount of cancer cells per unit of volume (e.g., 1 mL) or other measured unit (e.g., per unit field in the case of a histological analysis) is quantitated. The cancer cell population, in certain embodiments, can be determined as a percentage of the total blood cells. In other cases, the specimen extracted from the patient is a tissue specimen (e.g., a biopsy extracted from suspected cancerous tissue), where the number or amount of cancer cells can be measured, for example, on the basis of the number or amount of cancer cells per unit weight of the tissue. The number or amount of cancer cells in the extracted specimen can be compared with the numbers or amounts of cancer cells measured in reference samples to assess the efficacy of the regimen and amelioration of the cancer under therapy. For example, the reference sample is a specimen extracted from the patient undergoing therapy, wherein the specimen from the patient is extracted at an earlier time point (e.g., prior to receiving the regimen, as a baseline reference sample, or at an earlier time point while receiving the therapy). In another example, the reference sample is extracted from a healthy, noncancer-afflicted patient.

In other cases, the cancer cell population in the extracted specimen can be compared with a predetermined reference range. In a specific embodiment, the predetermined reference range is based on the number or amount of cancer cells obtained from a population(s) of patients suffering from the same type of cancer as the patient undergoing the therapy.

### Pharmaceutical Therapeutics

For therapeutic uses, the compositions or agents described herein may be administered systemically, for example, formulated in a pharmaceutically-acceptable buffer such as physiological saline. Preferable routes of administration include, for example, subcutaneous, intravenous, intraperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

### Formulation of Pharmaceutical Compositions

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16, or 18 mg/Kg body weight. Of course, this dosage amount may be adjusted upward or downward, as is routinely done in such treatment protocols, depending on the results of the initial clinical trials and the needs of a particular patient.

In some cases, the compound or composition of the invention is administered at a dose that is lower than the human equivalent dosage (HED) of the no observed adverse effect level (NOAEL) over a period of three months, four months, six months, nine months, 1 year, 2 years, 3 years, 4 years, or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs, or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005, incorporated herein by reference.

The amount of a compound of the invention used in the prophylactic and/or therapeutic regimens which will be effective in the prevention, treatment, and/or management of cancer can be based on the currently prescribed dosage of the compound as well as assessed by methods disclosed herein and known in the art. The frequency and dosage will vary also according to factors specific for each patient depending on the specific compounds administered, the severity of the cancerous condition, the route of administration, as well as age, body, weight, response, and the past medical history of the patient. For example, the dosage of a compound of the invention which will be effective in the treatment, prevention, and/or management of cancer can be determined by administering the compound to an animal model such as, e.g., the animal models disclosed herein or known to those skilled in the art. In addition, in vitro assays may optionally be employed to help identify optimal dosage ranges.

In some aspects, the prophylactic and/or therapeutic regimens comprise titrating the dosages administered to the patient so as to achieve a specified measure of therapeutic efficacy. Such measures include a reduction in the cancer cell population in the patient. In certain cases, the dosage of the compound of the invention in the prophylactic and/or therapeutic regimen is adjusted so as to achieve a reduction in the number or amount of cancer cells found in a test specimen extracted from a patient after undergoing the prophylactic and/or therapeutic regimen, as compared with a reference sample. Here, the reference sample is a specimen extracted from the patient undergoing therapy, wherein the specimen is extracted from the patient at an earlier time point. In one aspect, the reference sample is a specimen extracted from the same patient, prior to receiving the prophylactic and/or therapeutic regimen. For example, the number or amount of cancer cells in the test specimen is at least 2%, 5%, 10%, 15%, 20%, 30%, 40%, 50%, 60%, 70%, 80%, 90%, 95%, or 99% lower than in the reference sample.

In some cases, the dosage of the compound of the invention in the prophylactic and/or therapeutic regimen is adjusted so as to achieve a number or amount of cancer cells that falls within a predetermined reference range. In these embodiments, the number or amount of cancer cells in a test specimen is compared with a predetermined reference range.

In other embodiments, the dosage of the compound of the invention in prophylactic and/or therapeutic regimen is adjusted so as to achieve a reduction in the number or amount of cancer cells found in a test specimen extracted from a patient after undergoing the prophylactic and/or therapeutic regimen, as compared with a reference sample, wherein the reference sample is a specimen is extracted from a healthy, noncancer-afflicted patient. For example, the number or amount of cancer cells in the test specimen is at least within 60%, 50%, 40%, 30%, 20%, 15%, 10%, 5%, or 2% of the number or amount of cancer cells in the reference sample.

In treating certain human patients having solid tumors, extracting multiple tissue specimens from a suspected tumor site may prove impracticable. In these cases, the dosage of the compounds of the invention in the prophylactic and/or therapeutic regimen for a human patient is extrapolated from doses in animal models that are effective to reduce the cancer population in those animal models. In the animal models, the prophylactic and/or therapeutic regimens are adjusted so as to achieve a reduction in the number or amount of cancer cells found in a test specimen extracted from an animal after undergoing the prophylactic and/or therapeutic regimen, as compared with a reference sample. The reference sample can be a specimen extracted from the same animal, prior to receiving the prophylactic and/or therapeutic regimen. In specific embodiments, the number or amount of cancer cells in the test specimen is at least 2%, 5%, 10%, 15%, 20%, 30%, 40%, 50%, or 60% lower than in the reference sample. The doses effective in reducing the number or amount of cancer cells in the animals can be normalized to body surface area (e.g., mg/m²) to provide an equivalent human dose.

The prophylactic and/or therapeutic regimens disclosed herein comprise administration of compounds of the invention or pharmaceutical compositions thereof to the patient in a single dose or in multiple doses (e.g., 1, 2, 3, 4, 5, 6, 7, 8, 10, 15, 20, or more doses).

In one aspect, the prophylactic and/or therapeutic regimens comprise administration of the compounds of the invention or pharmaceutical compositions thereof in multiple doses. When administered in multiple doses, the compounds or pharmaceutical compositions are administered with a frequency and in an amount sufficient to prevent, treat, and/or manage the condition. For example, the frequency of administration ranges from once a day up to about once every eight weeks. In another example, the frequency of administration ranges from about once a week up to about once every six weeks. In another example, the frequency of administration ranges from about once every three weeks up to about once every four weeks.

Generally, the dosage of a compound of the invention administered to a subject to prevent, treat, and/or manage cancer is in the range of 0.01 to 500 mg/kg, e.g., in the range of 0.1 mg/kg to 100 mg/kg, of the subject's body weight. For example, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight. In another example, the dosage of a compound of the invention administered to a subject to prevent, treat, and/or manage cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight.

In another example, the dosage of a compound of the invention administered to a subject to prevent, treat, and/or manage cancer in a patient is a unit dose of 0.1 to 50 mg, 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In another example, the dosage of a compound of the invention administered to a subject to prevent, treat, and/or manage cancer in a patient is in the range of 0.01 to 10 g/m², and more typically, in the range of 0.1 g/m² to 7.5 g/m², of the subject's body weight. For example, the dosage administered to a subject is in the range of 0.5 g/m² to 5 g/m², or 1 g/m² to 5 g/m² of the subject's body's surface area.

In another example, the prophylactic and/or therapeutic regimen comprises administering to a patient one or more doses of an effective amount of a compound of the invention, wherein the dose of an effective amount achieves a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention.

In another example, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 day, 1 month, 2 months, 3 months, 4 months, 5 months, 6 months, 7 months, 8 months, 9 months, 10 months, 11 months, 12 months, 15 months, 18 months, 24 months, or 36 months. In other embodiments, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 day, 1 month, 2 months, 3 months, 4 months, 5 months, 6 months, 7 months, 8 months, 9 months, 10 months, 11 months, 12 months, 15 months, 18 months, 24 months, or 36 months.

### Combination Therapy

In one example, the active compounds are administered in combination therapy, i.e., combined with other agents, e.g., therapeutic agents, that are useful for treating pathological conditions or disorders, such as various forms of cancer. The term “in combination” in this context means that the agents are given substantially contemporaneously, either simultaneously or sequentially. If given sequentially, at the onset of administration of the second compound, the first of the two compounds is in some cases still detectable at effective concentrations at the site of treatment.

The administration of a compound or a combination of compounds for the treatment of a neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Accordingly, in some examples, the prophylactic and/or therapeutic regimen comprises administration of a compound of the invention in combination with one or more additional anticancer therapeutics. In one example, the dosages of the one or more additional anticancer therapeutics used in the combination therapy is lower than those which have been or are currently being used to prevent, treat, and/or manage cancer. The recommended dosages of the one or more additional anticancer therapeutics currently used for the prevention, treatment, and/or management of cancer can be obtained from any reference in the art including, but not limited to, Hardman et al., eds., Goodman & Gilman's The Pharmacological Basis Of Basis Of Therapeutics, 10th ed., McGraw-Hill, New York, 2001; Physician's Desk Reference (60^{th} ed., 2006), which is incorporated herein by reference in its entirety.

The compound of the invention and the one or more additional anticancer therapeutics can be administered separately, simultaneously, or sequentially. In various aspects, the compound of the invention and the additional anticancer therapeutic are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In another example, two or more anticancer therapeutics are administered within the same patient visit.

In certain aspects, the compound of the invention and the additional anticancer therapeutic are cyclically administered. Cycling therapy involves the administration of one anticancer therapeutic for a period of time, followed by the administration of a second anticancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In another example, the anticancer therapeutics are administered concurrently to a subject in separate compositions. The combination anticancer therapeutics of the invention may be administered to a subject by the same or different routes of administration.

When a compound of the invention and the additional anticancer therapeutic are administered to a subject concurrently, the term “concurrently” is not limited to the administration of the anticancer therapeutics at exactly the same time, but rather, it is meant that they are administered to a subject in a sequence and within a time interval such that they can act together (e.g., synergistically to provide an increased benefit than if they were administered otherwise). For example, the anticancer therapeutics may be administered at the same time or sequentially in any order at different points in time; however, if not administered at the same time, they should be administered sufficiently close in time so as to provide the desired therapeutic effect, preferably in a synergistic fashion. The combination anticancer therapeutics of the invention can be administered separately, in any appropriate form and by any suitable route. When the components of the combination anticancer therapeutics are not administered in the same pharmaceutical composition, it is understood that they can be administered in any order to a subject in need thereof. For example, a compound of the invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various aspects, the anticancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart. In one example, the anticancer therapeutics are administered within the same office visit. In another example, the combination anticancer therapeutics of the invention are administered at 1 minute to 24 hours apart.

### Release of Pharmaceutical Compositions

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level. Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Kits or Pharmaceutical Systems

The present compositions may be assembled into kits or pharmaceutical systems for use in ameliorating a neoplasia. Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

Targeting the Transcription Factor NF-κB with Harmine

The transcription factor, NF-κB, regulates genes that control a range of cellular functions including proliferation, survival, and release of cytokines and chemokines. Consequently, increased or inappropriate activation of NF-κB is found frequently in cancer, inflammatory conditions, and auto-immune diseases. Despite this prominent role in the pathogenesis of a diversity of human diseases, prior to the invention described herein, compounds that directly inhibit NF-κB had yet to be clinically developed.

As described in the examples that follow, in order to identify compounds that could specifically block the effect of NF-κB on gene expression, a cell line that produces the light-emitting enzyme, luciferase, when NF-κB is activated was generated. These cells were used to screen a library of natural products and other bioactive molecules to identify compounds that specifically inhibit NF-κB activity. From this approach, the natural product, harmine, was identified as an effective and specific inhibitor of NF-κB. In laboratory experiments, it was demonstrated that harmine (but not structurally modified forms of this compound) block NF-κB-dependent gene expression. Furthermore, when used alone or in conjunction with other therapies, harmine exerts anti-cancer effects through this mechanism.

Although NF-κB has been recognized as an important therapeutic target, prior to the invention described herein, as a transcription factor, it is not easy to inhibit its function with small organic molecules. As described herein, the identification of harmine as a potent and specific NF-κB inhibitor is useful for developing therapeutic uses of this compound. Harmine and the plant from which it is derived, the Syrian rue, have been used safely by native cultures for many years, indicating this method of inhibiting NF-κB is unlikely to be associated with major side effects. In addition, since harmine inhibits NF-κB function by a new mechanism, it also raises opportunities for medicinal chemistry approaches to develop even more effective NF-κB inhibitors based on this compound. Finally, there may be synergies between harmine and other treatments that indirectly affect NF-κB function (like tumor necrosis factor inhibitors including infliximab (Remicade®), adalimumab (Humira®), certolizumab pegol (Cimzia®), golimumab (Simponi®), and etanercept (Enbrel®)).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Materials and Methods

### Cell Culture and Reagents

OVCAR8 and OVKATE cells and SK-BR-3 cells were grown in RPMI containing 10% fetal bovine serum (FBS). MDA-MB-468 cells, STAT3-Luc cells, NF-κB-Luc cells (Nelson et al., 2008 Blood, 112: 5095-5102, incorporated herein by reference), and HeLa p65-EGFP cells (Lee et al., 2014 Mol Cell, 53(6):867-79) were maintained in DMEM with 10% FBS. U266 and INA-6 cells were obtained and maintained as described (Nelson et al., 2008 Blood, 112: 5095-5102). Cell lines were authenticated by short tandem repeat analysis. Cells were stimulated with 10 ng/ml IL-6, 10 ng/ml tumor necrosis factor (TNF), or 10 ng/ml interferon gamma (IFNγ) (Peprotech, Rocky Hill, N.J.); 10 ng/ml leukemia inhibitory factor (LIF; Calbiochem, Temecula, Calif.), and inhibitors used included Jak Inhibitor 1 (EMD, Billerica, Mass.), nifuroxazide (Chembridge, San Diego, Calif.), INDY, pimozide, pyrimethamine, harmine, and harmane (Sigma, St. Louis, Mo.), and TG101348 (VWR, Radnor, Pa.).

### Immunoblotting and Cellular Fractionation

Immunoblots were performed as described (Nelson et al., 2008 Blood, 112: 5095-5102). Nuclear and cytoplasmic fractions were isolated using the Nuclear Extract Kit (Active Motif North America, Carlsbad, Calif.). Antibodies were used recognizing phospho-tyrosine STAT3 (9131), PARP (9542), p65 (8242), RelB (4922) (Cell Signaling; Beverly, Mass.); actin (A-5316) and tubulin (T-5168) (Sigma); and STAT3 (sc-482), p50 (sc-7178), and calnexin (sc-11397) (Santa Cruz Biotechnology; Santa Cruz, Calif.).

Short Interfering RNA (siRNA)

Cells were reverse transfected with 10 nM STAT3 siRNA (pool, #2, and #3), PTP6N siRNA, STAT5a siRNA, BCL3 siRNA or Jak2 siRNA (Dharmacon, Thermo Scientific, Lafayette, Colo.), NF-κB p65 siRNA (#1, Cell Signaling), RelB siRNA (Santa Cruz Biotechnology), or Control #3 siRNA (Dharmacon) using Lipofectamine RNA interference (RNAi) Max (Invitrogen, Carlsbad, Calif.) following the protocol from Dharmacon. Cells were harvested 2 or 3 days after transfection.

For rescue experiments, OVCAR8 cells were reverse transfected with siRNA to STAT3 or control siRNA. The following day, cells were transfected with control (pcDNA3-EGFP) (Addgene plasmid #13031) or RelB cFlag pcDNA3 (Addgene plasmid #20017) for 24 hours, after which mRNA was isolated.

### Dual Luciferase Reporter Assay

Cells were transfected with firefly luciferase plasmids for STAT3 (m67-Luc) or NF-KB-Luc (Stratagene, Santa Clara, Calif.) and a Renilla luciferase plasmid (Promega, Madison, Wis.). The NF-κB-responsive cell line was generated with an NF-κB-responsive reporter plasmid (catalogue no. 219078; Strategene, La Jolla, Calif., incorporated herein by reference). Cells were also co-transfected with a plasmid expressing Renilla luciferase under a constitutive promoter for normalization (pRL-TK plasmid; Promega; Madison, Wis., incorporated herein by reference). Dual luciferase assays were performed as described (Walker et al., 2007 Oncogene, 26: 224-233, incorporated herein by reference).

### Gene Expression Analysis

RNA was isolated using an RNeasy kit (Qiagen, Valencia, Calif.). cDNA was generated using the Taqman reverse transcription kit (Applied Biosystems, Foster City, Calif.). qRT-PCR was performed using Sybr Select or Power Sybr green master mix (Applied Biosystems). Samples were plated in triplicate and run on a 7300 or 7500 real time PCR machine (Applied Biosystems), using the indicated primers (Table 2). Target gene expression was normalized to actin, HPRT, or GAPDH. Data are expressed as mean fold change+/−SEM and are representative of at least two independent biological replicates.

TABLE 2
Primers sequences for qRT-PCR

| | | |
|---|---|---|
| Actin | TCCCTGGAGAAGAGCTACGA | AGCACTGTGTTGGCGTACAG |
| | (SEQ ID NO: 10) | (SEQ ID NO: 11) |
| HPRT | GAACGTCTTGCTCGAGATGTG | CCAGCAGGTCAGCAAAGAATT |
| | (SEQ ID NO: 12) | (SEQ ID NO: 13) |
| GAPDH | AATCCCATCACCATCTTCCA | TGGACTCCACGACGTACTCA |
| | (SEQ ID NO: 14) | (SEQ ID NO: 15) |
| TNFAIP3 | CCTTGGAAGCACCATGTTTG | TTGTGTGGTTCGAGGCACAT |
| | (SEQ ID NO: 16) | (SEQ ID NO: 17) |
| BIRC3 | GGGAAGAGGAGAGAGAAAGAGC | TCCAGGATTGGAATTACACAAG |
| | (SEQ ID NO: 18) | (SEQ ID NO: 19) |
| IL8 | TCCTGATTTCTGCAGCTCTGT | AATTTCTGTGTTGGCGCAGT |
| | (SEQ ID NO: 20) | (SEQ ID NO: 21) |
| RelA | TCTGCTTCCAGGTGACAGTG | ATCTTGAGCTCGGCAGTGTT |
| | (SEQ ID NO: 22) | (SEQ ID NO: 23) |
| RelB | AGCATCCTTGGGGAGAGC | AGGCAGTCACCTCCACCTC |
| | (SEQ ID NO: 24) | (SEQ ID NO: 25) |
| STAT3 | GAGAAGCCAATGGAGATTGC | GACATCCTGAAGGTGCTGCT |
| | (SEQ ID NO: 26) | (SEQ ID NO: 27) |
| BCL3 | CCTCTGGTGAACCTGCCTAC | TACCCTGCACCACAGCAATA |
| | (SEQ ID NO: 28) | (SEQ ID NO: 29) |
| BCLX | GGTATTGGTGAGTCGGATCG | TGCTGCATTGTTCCCATAGA |
| | (SEQ ID NO: 30) | (SEQ ID NO: 31) |
| SOCS3 | TCAAGACCTTCAGCTCCAAG | TGACGCTGAGCGTGAAGAAG |
| | (SEQ ID NO: 32) | (SEQ ID NO: 33) |

### Gene Set Enrichment Analysis (GSEA)

Data were downloaded from Gene Expression Omnibus (GEO) and the The Cancer Genome Atlas (TCGA). To detect the presence of the NF-κB gene signature, data from STAT3 inhibition was compared to control and GSEA (Subramanian et al., 2005 Proc Natl Acad Sci U.S.A., 102(43):15545-50; Mootha et al., 2003 Nat Genet, 34(3):267-73) was run to identify the Hallmark signatures (version 5.1) (which includes an NF-κB gene signature) that were enriched in the STAT3 inhibition gene sets (GSE70115 (Cuenca-Lopez et al., 2015 Oncotarget, 6(29):27923-37), GSE47763 (Timme et al., 2014 Oncogene, 33(25):325666), GSE31534 (Wang et al., 2012 PLoS One, 7(4):e34247), and GSE68826). To detect the presence of a STAT3 gene signature, gene expression datasets (TCGA ovarian (Cancer Genome Atlas Research N, 2011 Nature, 474(7353):609-15) and GSE2912 (Agnelli et al., 2005 J Clin Oncol, 23(29):7296-306) were stratified based on RelB expression (top 150 compared to bottom 150 of ovarian tumors; top 10 to the bottom 10 of multiple myeloma tumors) and GSEA was performed using the STAT3 signature defined by (Walker et al., 2016 Blood, 127(7):948-51).

### Single Cell Nuclear Translocation Analysis

Hela cells stably expressing EGFP-tagged p65 were imaged for one hour prior to addition of either 1 uM Jak inhibitor 1 or 100 ng/mL TNF, and then for an additional 18 hours. Transmitted light and widefield epifluorescent images were captured at 10 min intervals using a BD Pathway 855 Bioimager with a 20× objective (0.75 NA; Olympus). Imaging was performed in an environmental chamber set to 37θC and 5% CO₂. Five fields of view were analyzed per condition and the nuclear translocation of p65-EGFP was manually scored for each cell.

### Chromatin Immunoprecipitation

SKBR3 cells stimulated with IL-6 for 30 minutes, OVCAR8 cells treated with harmine for 16 hours and then stimulated with TNF for 30 minutes, or U266 cells treated with Jak inhibitor 1 for 3 hours were analyzed by ChIP. Cross-linking was performed with 1% formaldehyde (0.37% for U266 cells) for 10 min at room temperature, followed by quenching of formaldehyde using 0.125 M glycine. ChIP was performed essentially as described (Nelson et al., 2004 J Biol Chem, 279(52):54724-30). Chromatin was sheared by sonication using a Qsonica Q700 sonicator with a microtip at approximately 75% amplitude. Anti-STAT3 antibody as described above, anti-p65 (Santa Cruz Biotechnology, sc-109), or anti-RNA polymerase II (Santa Cruz Biotechnology, sc-9001) were used. ChIP product was analyzed by qPCR using the following primers: RELB (CAACCTCTCGATCCTGAAGC (SEQ ID NO: 34) and ATCACGCCTTACCCATTGAG (SEQ ID NO: 35)), IL8 (GAAAACTTTCGTCATACTCCG (SEQ ID NO: 36) and GAAAGTTTGTGCCTTATGGAG (SEQ ID NO: 37)), BIRC3 (CACGAGCAATGAAGCAAATG (SEQ ID NO: 38) and GTGCACTGGTGCTTTCCTTT (SEQ ID NO: 39)), TNFAIP3 (CTATAATTTGCGCCGCTGAC (SEQ ID NO: 40) and TTTCCTTGGGTCATTGACTTT (SEQ ID NO: 41)). Data were normalized relative to input and a nonbinding region of the rhodopsin (RHO) gene (Walker et al., 2013 Mol Cell Biol, 33(15):2879-90).

### Identification of NF-κB Inhibitors

To identify NF-κB inhibitors, the Prestwick Chemical Library, which contains 1120 bioactive compounds, was screened for activity against cells expressing NF-κB-dependent luciferase (Nelson et al., 2008 Blood, 112(13):5095-102). To confirm the specificity of identified compounds, NF-κB-Luc and STAT3-Luc cells were treated with the indicated doses of drug for one hour followed by stimulation with TNF or IL-6 respectively for 6 hours. Luciferase activity was assessed using the Bright-Glo Luciferase Assay system (Promega).

### Quantitation of Viable Cell Number

Cells were treated with the indicated drugs in 96 well plates for 48 to 72 hours. Viable cell number was assessed using ATP-dependent bioluminescence (Cell-TiterGlo; Promega). Data are expressed as average+/−SD of at least two replicates, and are representative of at least two separate experiments.

### Example 2: STAT3 Inhibition Results in NF-κB Activation

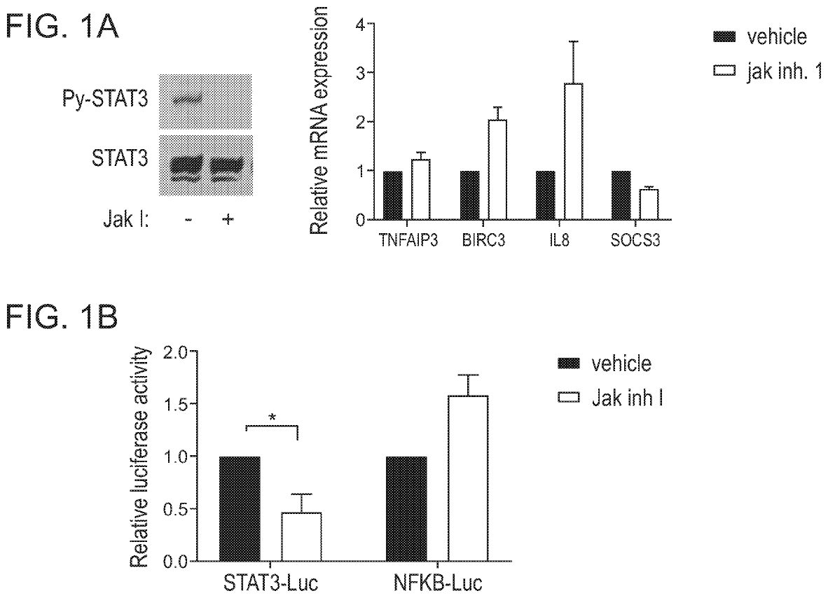

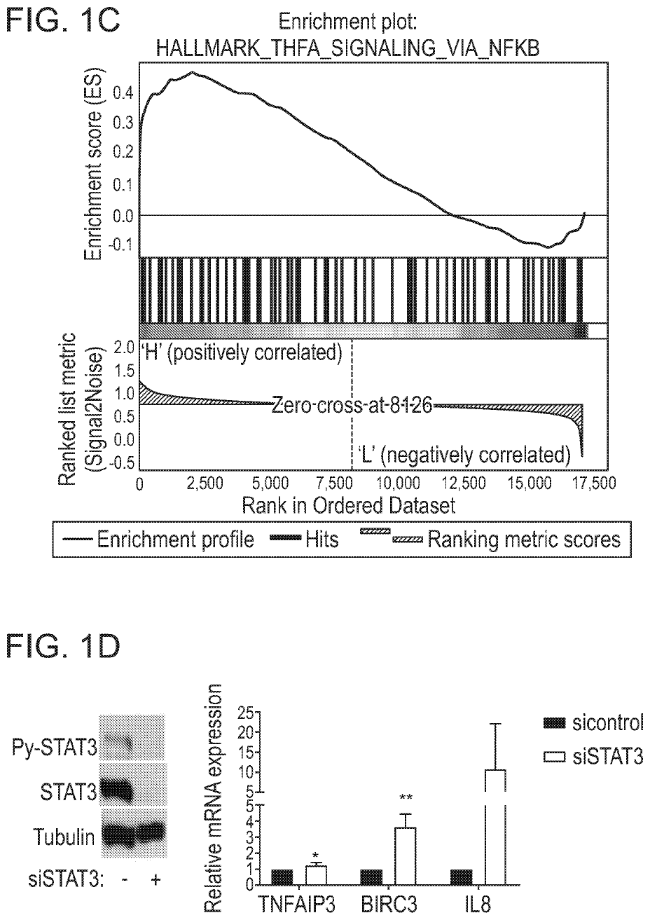

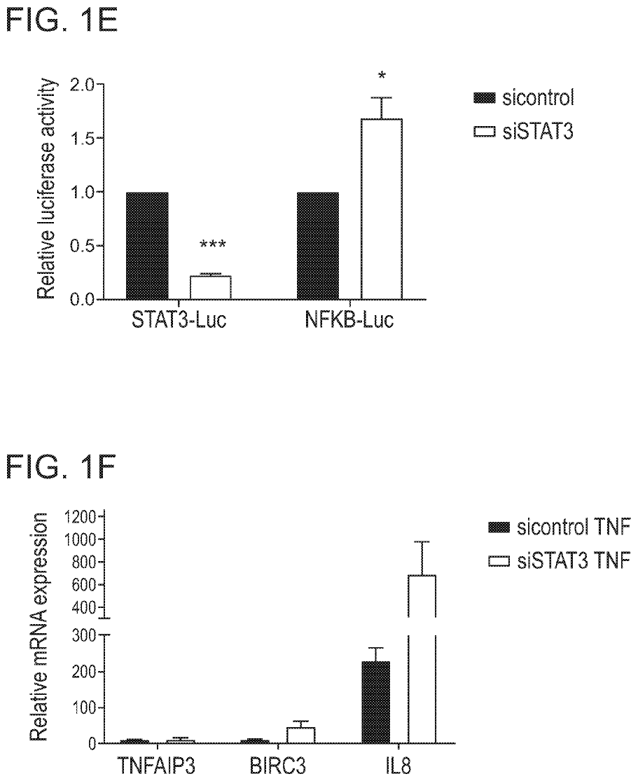

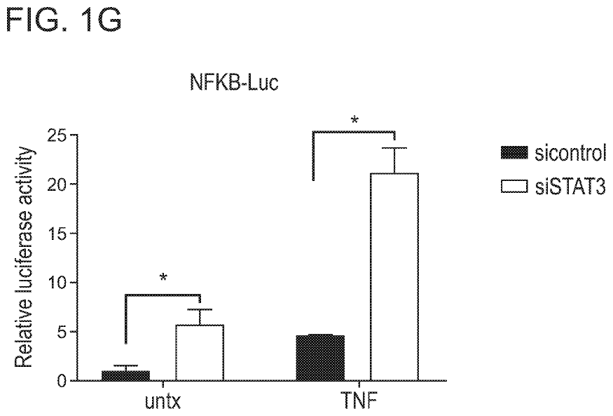

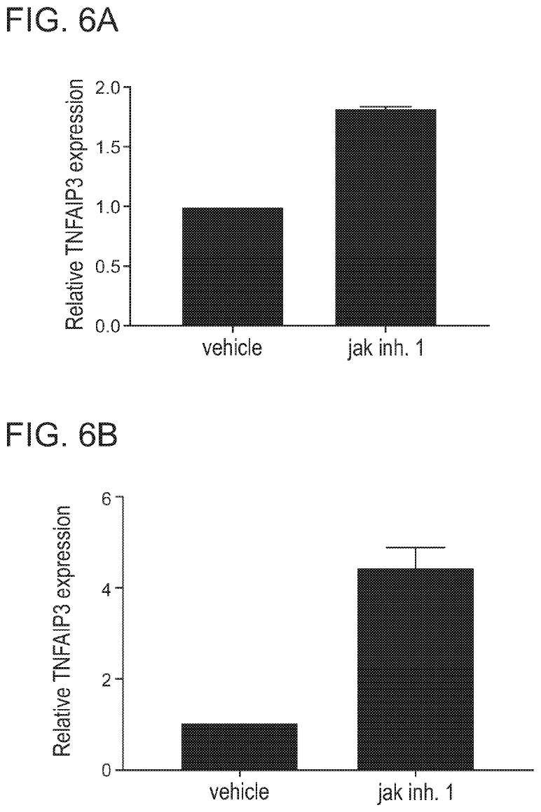

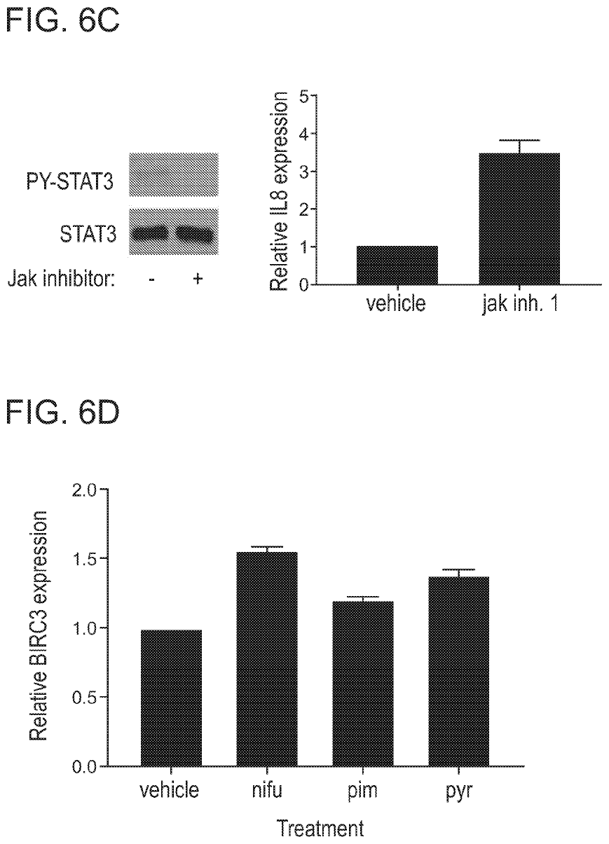

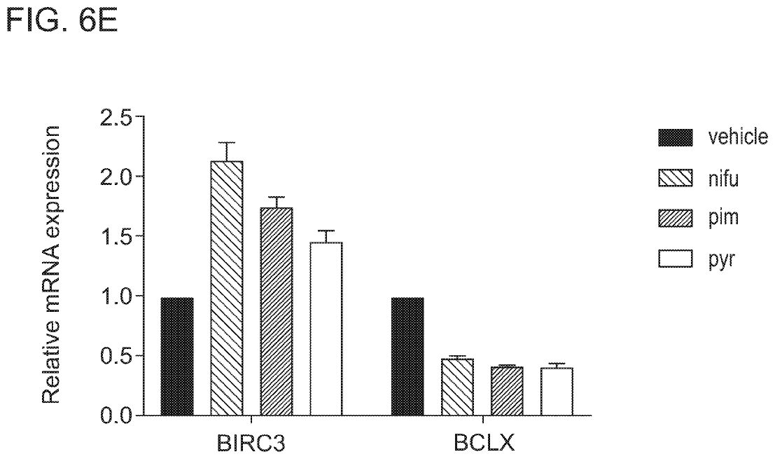

To anticipate potential limitations to STAT3-targeted therapy, the possibility that complementary transcription factor pathways would become activated upon STAT3 inhibition was considered. To test the hypothesis that STAT3 inhibitors would enhance NF-κB-dependent gene expression, cells containing constitutively activated STAT3 were treated with Jak inhibitor 1 to inhibit the activating tyrosine phosphorylation of STAT3. Then, expression of the well characterized NF-κB target genes TNFAIP3, BIRC3, and IL8 were analyzed (Krikos et al., 1992 J Biol Chem, 267(25):17971-6; Mukaida et al., 1990 J Biol Chem, 265(34):21128-33; Erl et al., 1999 Circ Res, 84(6):668-7732-34; FIG. 1A). In OVCAR8 ovarian cancer cells (FIG. 1A), MBA-MB-468 breast cancer cells (FIG. 6A), INA-6 MM cells (FIG. 6B), and A498 gastric cancer cells (FIG. 6C), that NF-κB target genes are enhanced upon STAT3 inhibition via Jak inhibition. To extend this finding, OVCAR8 and INA-6 cells were treated with three small molecule STAT3 inhibitors that have differing mechanisms of action: nifuroxazide (Nelson et al., 2008 Blood, 112(13):5095-102), pimozide (Nelson et al., 2012 Genes Cancer, 3(7-8):503-11; Nelson et al., 2011 Blood, 117(12):3421-9), and pyrimethamine (Takakura et al., 2011 Hum Mol Genet, 20(21):4143-54; Nelson et al., 2011 Oncotarget, 2(6):518-24). Treatment with all three STAT3 inhibitors similarly resulted in upregulation of NF-κB target gene expression (FIG. 6D and FIG. 6E). To rule out that this effect occurred only on select NF-κB target genes, two complementary approaches were used. First, the effects of STAT3 inhibition were analyzed on an NF-κB reporter construct that contains four NF-κB binding sites upstream of luciferase. It was identified that, consistent with effects on endogenous gene expression, NF-κB activity was enhanced upon STAT3 inhibition in OVCAR8 cells (FIG. 1B). Second, the presence of an NF-κB signature from transcriptomics datasets of cells treated with Jak inhibitors (thus inhibiting STAT3) was analyzed using GSEA. It was identified that inhibition of STAT3 resulted in significant enrichment of the NF-κB gene signature in both kidney and breast cancer cells (FIG. 1C and Table 1). As shown in Table 1, NF-κB target gene enrichment was determined by GSEA following STAT3 inhibition, using the indicated perturbations. NES, normalized enrichment score. Taken together, these findings suggest that a consequence of STAT3 inhibition is activation of the NF-κB signaling pathway.

TABLE 1
NF-κB target gene sets are enriched following STAT3 inhibition

| GEO | | | | |
|---|---|---|---|---|
| Accession | Study | NES | p-value | q-value |
| GSE68826 | IL-6 + Jak inhibition vs IL-6 in kidney cells | 1.988731 | <0.001 | 0.00102 |
| GSE70115 | Jak inhibition (and other kinases) vs vehicle in | 1.496059 | <0.001 | 0.0923803 |
| | breast cancer cells (Cuenca-Lopez, et al., 2015 | | | |
| | Oncotarget, 6(29): 27923-27937) | | | |
| GSE47763 | siSTAT3 vs control in esophageal carcinoma cells | 1.759301 | <0.001 | 0.0015406 |
| | (Timme et al., 2014 Oncogene, 33(25): 3256-3266) | | | |
| GSE31534 | siSTAT3 vs control in melanoma (Wang et al., 2012 | 1.70602 | <0.001 | 0.0041389 |
| | PLoS One, 7(4): e34247) | | | |

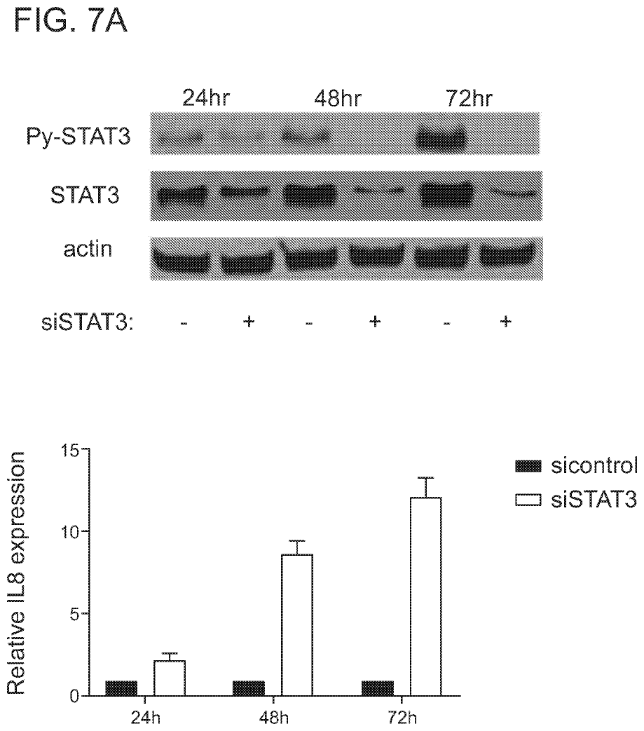

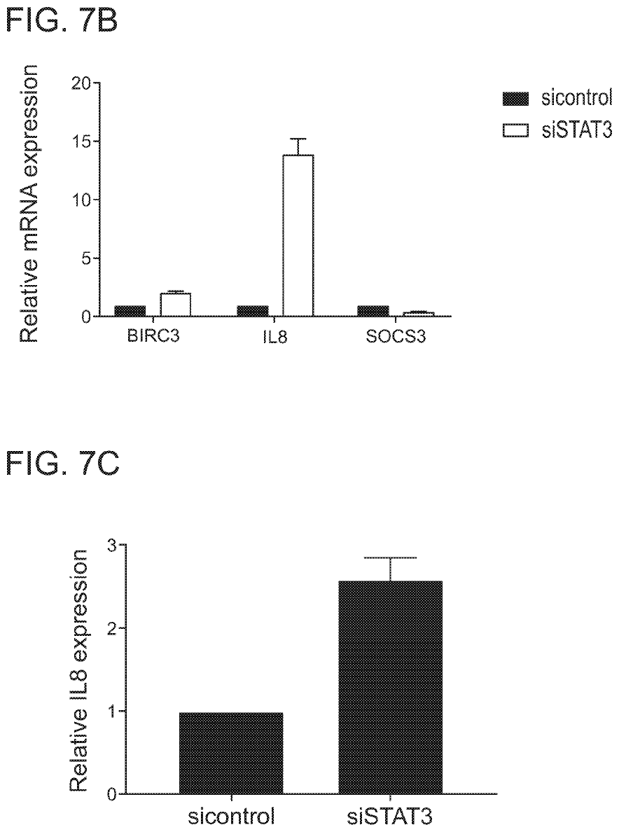

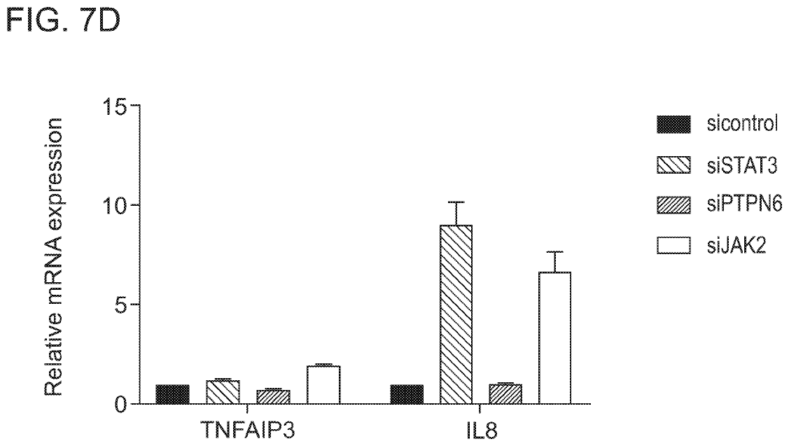

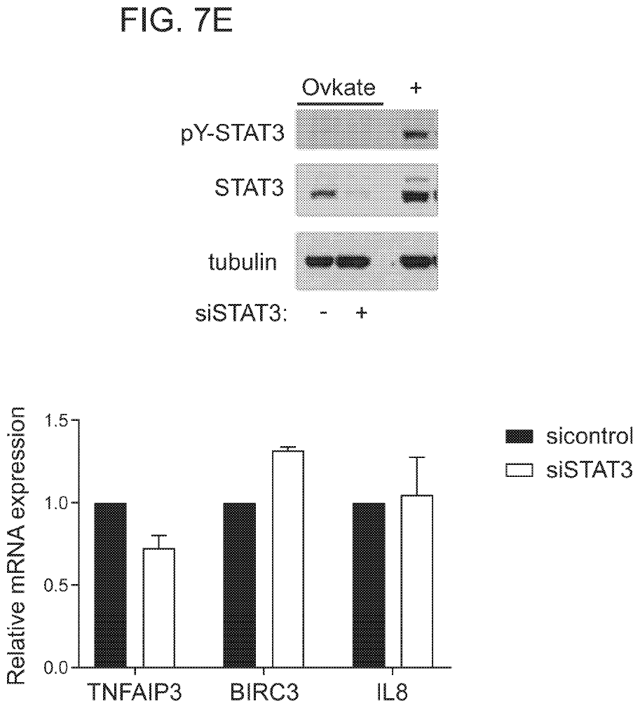

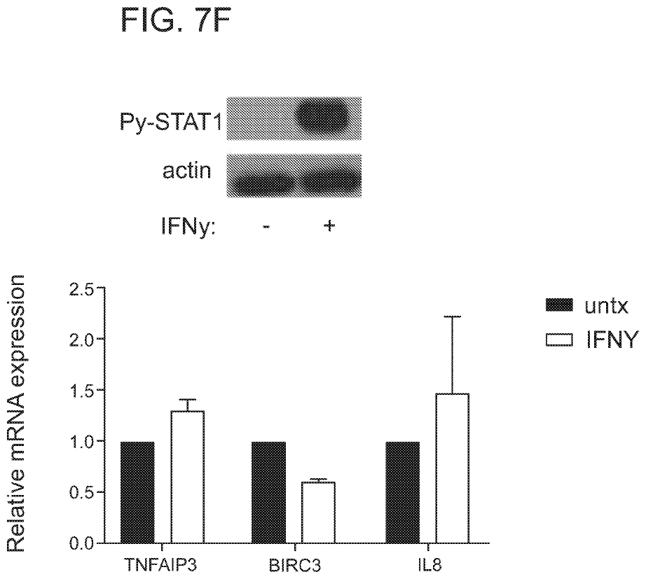

Because these drugs may have effects beyond STAT3 inhibition, it was next determined if inhibition of STAT3 by RNA interference (RNAi) would lead to a similar effect. It was identified that reducing the expression of STAT3 by siRNA (with either a pool or two distinct STAT3 siRNAs) resulted in upregulation of NF-κB target genes including TNFAIP3, BIRC3, and IL8 in OVCAR8 cells (FIG. 1D and FIG. 7A). Similar results were also observed in the breast cancer cell line, MDA-MB-468, and the gastric cancer cell line, A498 (FIG. 7B and FIG. 7C). Two additional lines of evidence suggest that STAT3 inhibition also affected the expression of other NF-κB target genes. First, it was identified that similar to STAT3 inhibition by drugs, knockdown of STAT3 by siRNA also resulted in increased expression of an NF-κB-regulated luciferase reporter (FIG. 1E). Second, it was identified that the NF-κB signature is significantly enriched in esophageal cancer and melanoma samples with reduced STAT3 expression (Table 1). Taken together, these findings demonstrate that STAT3 inhibition results in increased NF-κB activity in diverse cancer contexts.

Because NF-κB may become activated through toll-like receptors by dsRNA (Kumar et al., 1994 Proc Natl Acad Sci U.S.A., 91(14):6288-92), it was determined whether it was specifically the inhibition of STAT3 function, not the siRNA treatment itself, that led to enhanced NF-κB activity. It was identified that in OVCAR8 cells, siRNA to the upstream kinase Jak2, which also led to a loss of STAT3 activation, resulted in increased NF-κB target gene expression; however, siRNA targeting the phosphatase, PTPN6, did not result in the upregulation of NF-κB target gene expression (FIG. 7D). Moreover, siRNA to STAT3 in OVKATE cells, which lack significant STAT3 activation, had little effect on NF-κB target gene expression (FIG. 7E). Because siRNA treatment can also lead to activation of the interferon response pathway (Sledz et al., 2003 Nat Cell Biol, 5(9):834-9), it was examined whether IFN-γ treatment of OVCAR8 cells results in upregulation of NF-κB target genes. It was identified that treatment with IFN-γ resulted in STAT 1 activation and upregulation of STAT 1 target genes, as expected, but it did not result in significant upregulation of NF-κB target gene expression (FIG. 7F). Taken together, these findings further support the hypothesis that inhibition of STAT3 transcriptional function specifically enhances NF-κB target gene expression in cells that contain activated STAT3.

NF-κB transcriptional activity can be stimulated by a variety of factors often found in an inflammatory microenvironment, as may occur during cancer pathogenesis. Therefore, it was next examined whether inhibition of STAT3 would lead to enhanced NF-κB activity in the presence of cytokines such as TNF. It was identified that reducing STAT3 expression by siRNA resulted in enhanced TNF-induced NF-κB activity in OVCAR8 cells, as determined by NF-κB target gene expression (FIG. 1F) and NF-κB-dependent reporter assays (FIG. 1G). These data demonstrate that STAT3 inhibition amplifies the NF-κB activation response in cells treated with pro-inflammatory cytokines.

### Example 3: The NF-κB Subunit p65 is Necessary for Upregulation Upon STAT3 Inhibition

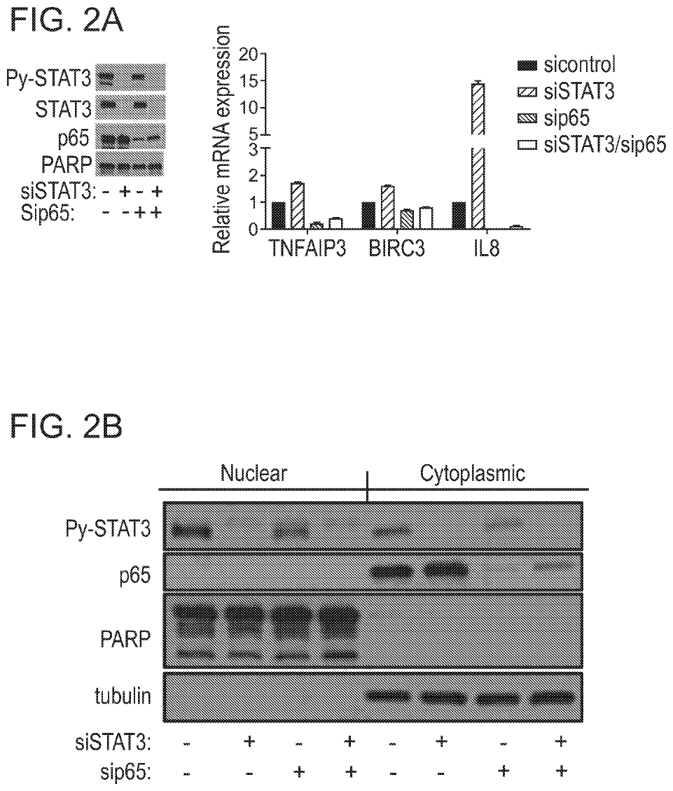

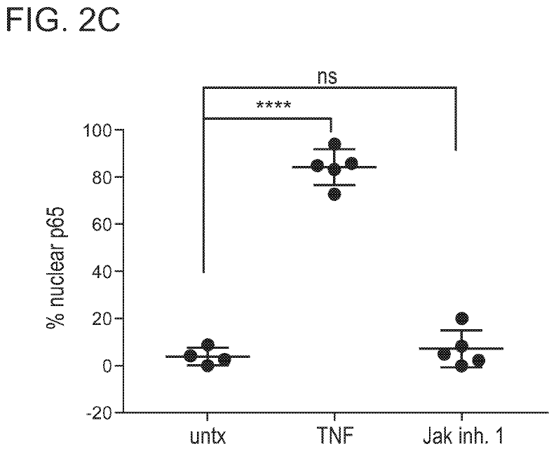

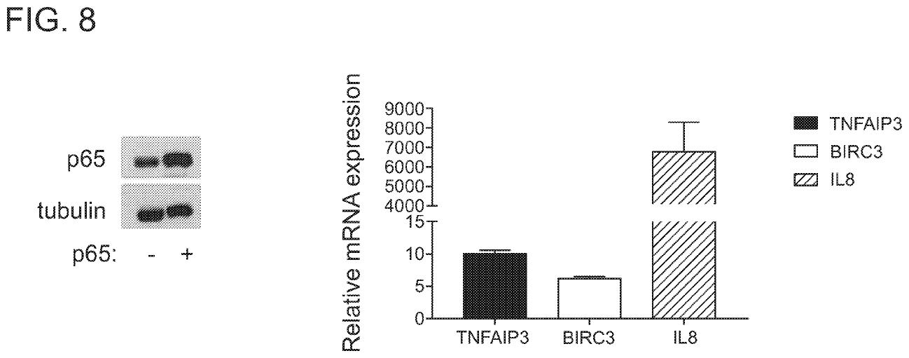

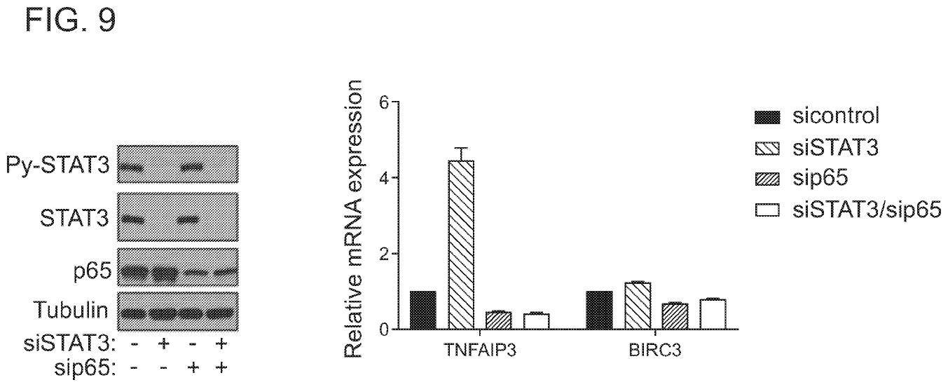

Having determined that inhibition of STAT3 results in upregulation of NF-κB activity, the molecular mechanism by which this occurs was investigated. It was identified that TNFAIP3, BIRC3, and IL8 are all upregulated by overexpression of the NF-κB subunit p65 in OVCAR8 cells (FIG. 8). This suggests that p65 may be the key mediator of the upregulation of NF-κB activity upon STAT3 inhibition. To determine if p65 is necessary for the increased expression of NF-κB target genes upon STAT3 inhibition, siRNA was used to knockdown expression of p65, alone or in combination with a knockdown of STAT3. It was identified that knockdown of p65 reduced the basal expression of all three NF-κB target genes in OVCAR8 cells (FIG. 2A and FIG. 9). Moreover, the upregulation of these genes upon STAT3 knockdown by two distinct siRNAs was completely abrogated when cells were also transfected with siRNA to p65 (FIG. 2A and FIG. 9). Taken together, these findings demonstrate that p65 is required for the upregulation of NF-κB activity upon STAT3 inhibition.

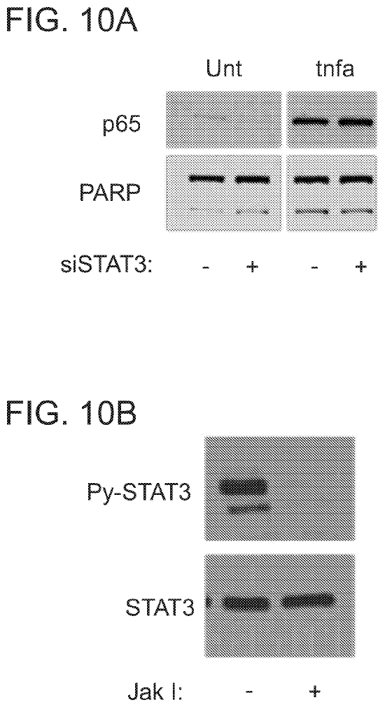

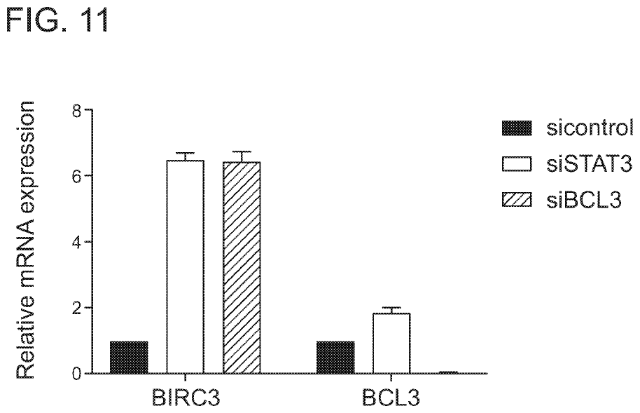

STAT3 has been reported to affect the activity of NF-κB in both positive and negative ways through alterations in subcellular localization, either by helping retain p65 in the nucleus (Lee et al., 2009 Cancer Cell, 15(4):28393) or by preventing p65 from accumulating in the nucleus (Grabner et al., 2015 Nat Commun, 6:6285). To determine if p65 nuclear translocation was affected by STAT3 inhibition, STAT3 was depleted by RNAi, and cellular fractionation was performed. It was identified that depletion of STAT3 in OVCAR8 cells had no effect on the nuclear levels of p65 under basal conditions (FIG. 2B) or following stimulation with TNF (FIG. 10A), suggesting that the increased activity of NF-κB may instead be due to modulation of p65 activity within the nucleus. To further evaluate this hypothesis, HeLa cells expressing p65 fused to EGFP (Lee et al., 2014 Mol Cell, 53(6):867-79) were used to examine the dynamics of p65 nuclear translocation at the single-cell level. When STAT3 was inhibited with Jak inhibitor 1 (FIG. 10B), p65 translocation was not significantly altered, in contrast to the distinctive translocation induced by TNF treatment (FIG. 2C). Thus, STAT3 does not affect p65 cellular localization.

### Example 4: STAT3 Upregulates Negative Regulators of NF-κB Transcriptional Function

Having determined that there was no significant change in p65 nuclear translocation with STAT3 inhibition, alternative mechanisms of NF-κB regulation were considered. BCL3 is a cofactor of NF-κB that can have both positive and negative effects on NF-κB activity. In addition, BCL3 is a known STAT3 target gene (Brocke-Heidrich et al., 2006 Oncogene, 25: 7297-7304). Therefore, it was determined if inhibition of BCL3 expression affected NF-κB activity. It was found that reduction of BCL3 expression by siRNA resulted in upregulation of NF-κB target genes (FIG. 9). While BCL3 expression was inhibited in some systems when STAT3 was inhibited, BCL3 expression was also increased in certain settings, likely reflecting the activation of NF-κB in these cells, because BCL3 is also regulated by p65 (Ge et al., 2003 Journal of Immunology, 171: 4210-4218). Therefore, while BCL3 may be linking STAT3 and NF-κB signaling in some systems, this does not explain the activity in all systems tested. Additional NF-κB subunits, such as p50 and p52, were also analyzed though they did not show significant similar data to reducing RelB expression. C-Rel was not assessed as it was not expressed in OVCAR8 cells.

The NF-κB subunit RelB has been shown to inhibit p65 activity when p65 and RelB heterodimerize in the nucleus (Marienfeld et al., 2003 J Biol Chem, 278(22):1985260). Therefore, the possibility that modulation of RELB by STAT3 might link the canonical (p65) and non-canonical (RelB) NF-κB pathways was examined. It was identified that RelB expression was reduced upon STAT3 inhibition by small molecule inhibitors or siRNA, measured both at the mRNA and protein levels (FIG. 3A). To determine if this relationship between STAT3 activation and RELB expression occurs in primary human tumors, gene expression data was analyzed from breast cancers in which STAT3 activation had been determined by immunohistochemistry to tyrosine phosphorylated STAT3 (Alvarez et al., 2005 Cancer Res, 65(12):5054-62) (GES5460). It was identified that RELB expression was significantly higher in breast tumors containing activated STAT3 compared to tumors without any STAT3 tyrosine phosphorylation (FIG. 3B). Moreover, gene set enrichment analysis in ovarian cancer (Cancer Genome Atlas Research N, 2011 Nature, 474(7353):609-15) and multiple myeloma (Agnelli et al., 2005 J Clin Oncol, 23(29):7296-306) patient samples that were stratified based on RELB expression showed enrichment of a STAT3 signature in samples with high RELB expression (FIG. 3C), further suggesting that RELB is positively correlated with STAT3 activation.

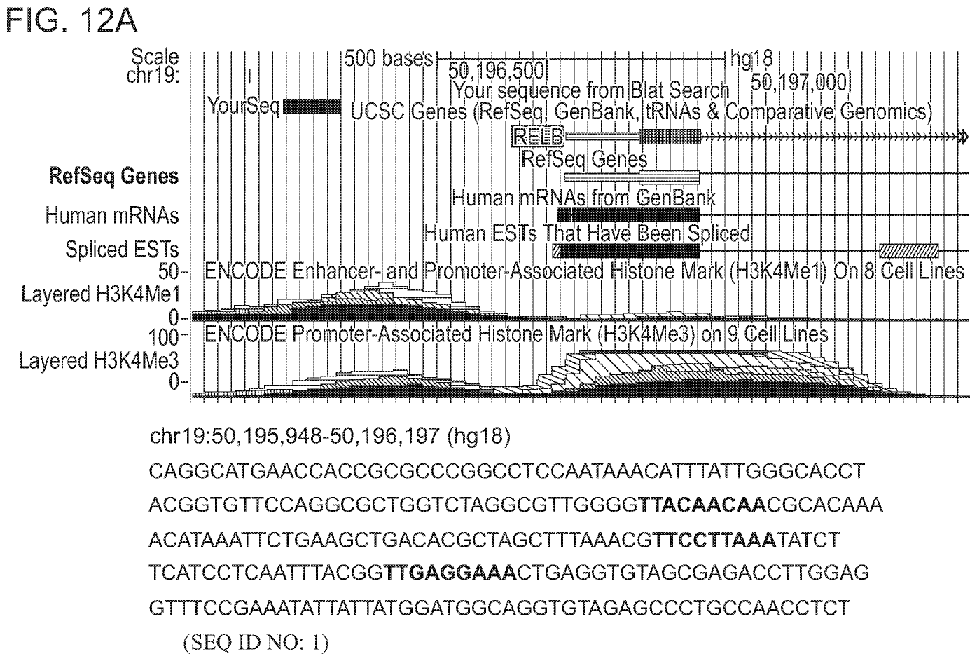

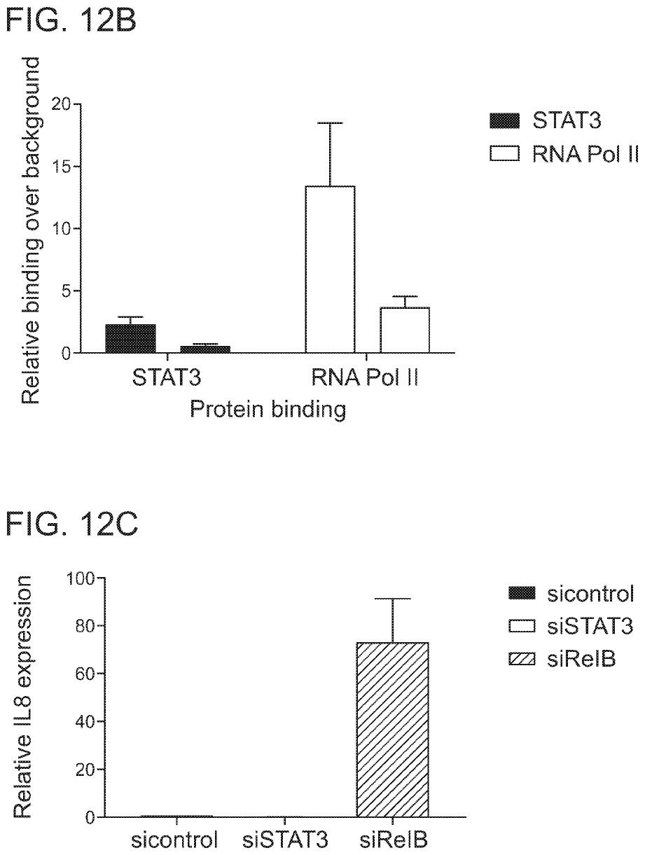

The associations identified between RELB expression and STAT3 activity both in cancer cell lines and patient samples raised the possibility that RELB expression is directly regulated by STAT3. Analysis of the promoter region upstream of the start of transcription of RELB identified three canonical STAT binding sites located within a stretch of 80 base pairs (FIG. 12). ChIP demonstrated that STAT3 binds to this region of the RELB promoter in SKBR3 cells when STAT3 is activated by LIF (FIG. 3D). In addition, upon small-molecule inhibition of STAT3, the binding of STAT3 and RNA polymerase II was reduced at this site in the STAT3-dependent U266 cells (FIG. 12B). This demonstrates that RELB is a direct target of STAT3, and raised the possibility that decreased RelB expression may mediate the increased activity of p65 upon STAT3 inhibition. To test this hypothesis, RelB was depleted by siRNA and upregulation of NF-κB target genes were identified (FIG. 3E). Consistent with its role as a negative regulator of NF-κB, knockdown of RelB resulted in upregulation of NF-κB target genes in OVCAR8 cells, which have constitutively active STAT3, and in SK-BR-3 cells, which lack strong STAT3 activation (Marotta et al., 2011 J Clin Invest, 121(7):2723-35). However, reduction of STAT3 expression only led to increased NF-κB target gene expression in OVCAR8 cells (FIG. 3E and FIG. 12C). Together, these findings demonstrate that STAT3 can upregulate RelB, a key negative regulator of the NF-κB pathway.

Given that STAT3 upregulates RelB, it was hypothesized that inhibition of STAT3 (and therefore loss of the expression of this negative regulator) would allow for enhanced activation of NF-κB activity by inflammatory cytokines, as might occur in the tumor milieu. It was identified that reducing the levels of RelB resulted in enhanced activation of NF-κB target genes upon TNF stimulation (FIG. 3F). These data further support the notion that RelB negatively regulates p65-mediated gene expression in physiologically-relevant systems.

To determine if RelB loss was necessary for the activation of NF-κB upon STAT3 inhibition, a rescue experiment was performed. After knockdown of STAT3, RelB was ectopically expressed, and then NF-κB-dependent gene expression was measured. Expression of RelB alone had no effect on the expression of the NF-κB target genes TNFAIP3 and IL-8; however, expression of RelB resulted in upregulation of BIRC3 expression (FIG. 3G). Importantly, re-expression of RelB after STAT3 inhibition resulted in the prevention of upregulation of the expression of TNFAIP3 and IL-8 suggesting that the loss of RelB expression by STAT3 inhibition is involved in the upregulation of NF-κB activity and increased expression of a subset of NF-κB target genes. Since BIRC3 was upregulated by both expression of RelB (FIG. 3G) and inhibition of RelB (FIG. 3E), it is likely that this gene has more complex regulation. Taken together, it was identified that STAT3 upregulates RelB, and that RelB loss is necessary for the activation of NF-κB upon STAT3 inhibition for at least some target genes.

### Example 5: Identification of Harmine as an Inhibitor of NF-κB

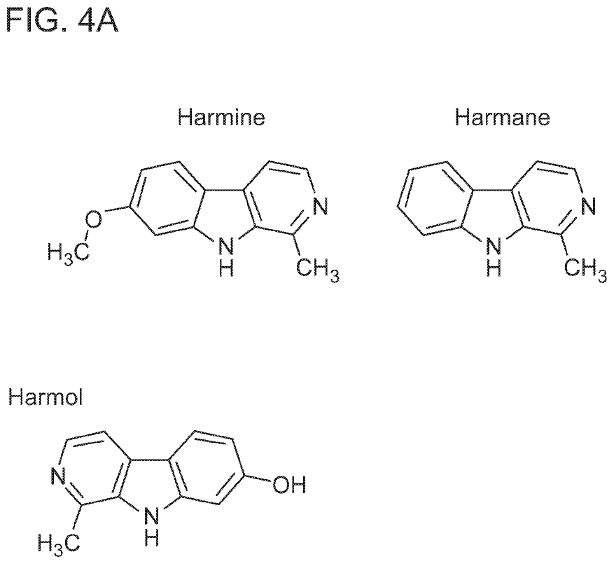

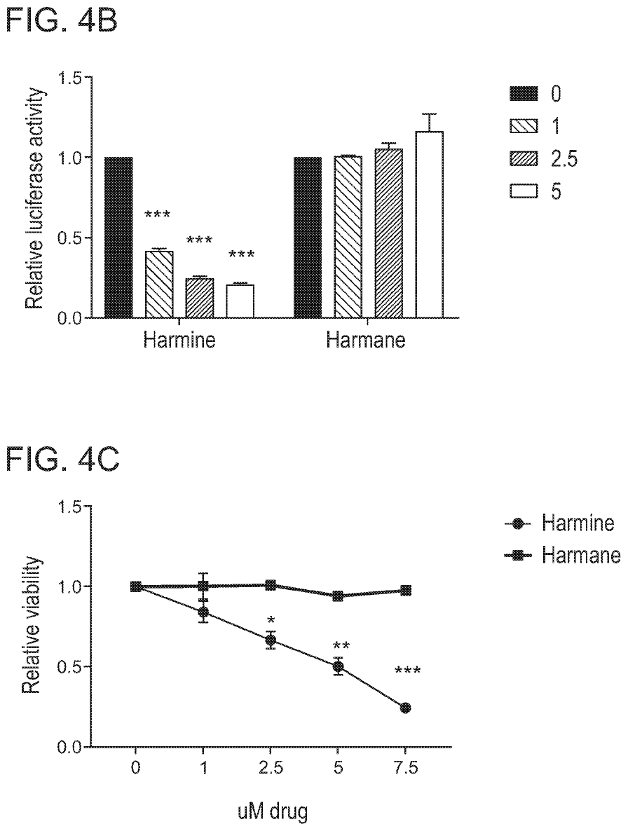

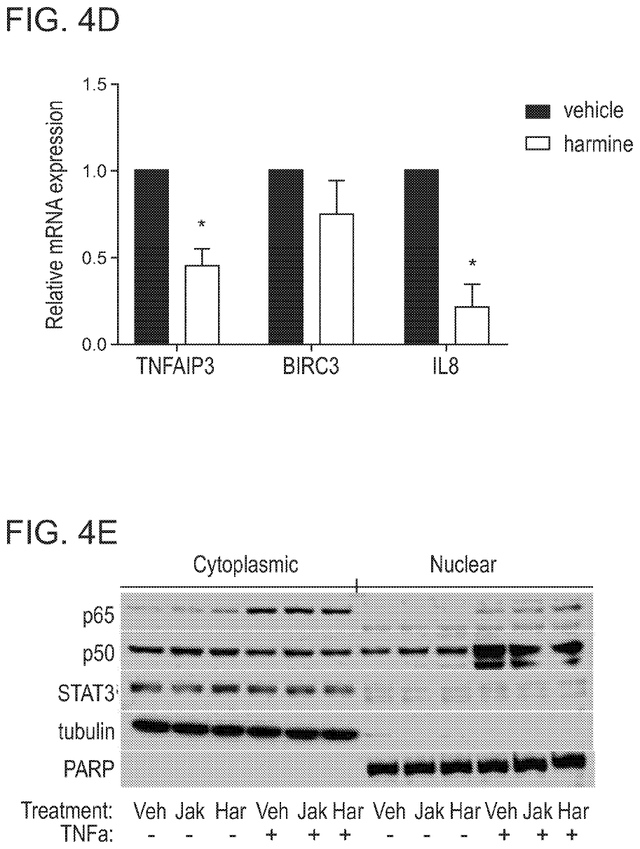

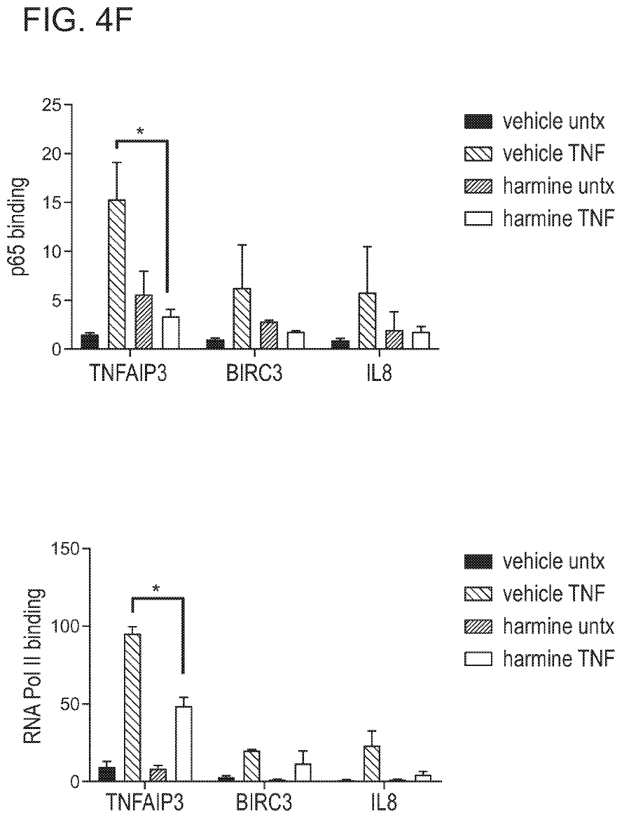

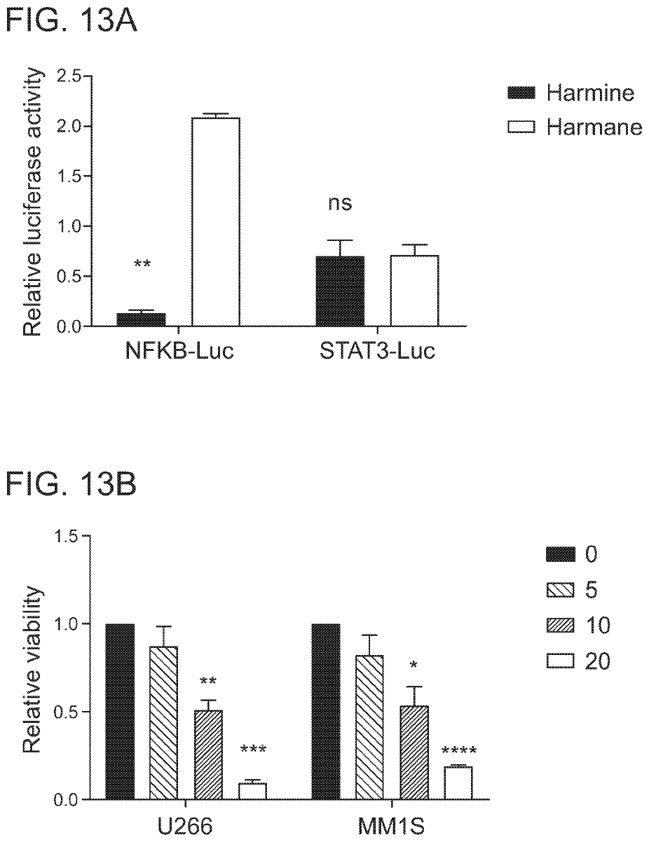

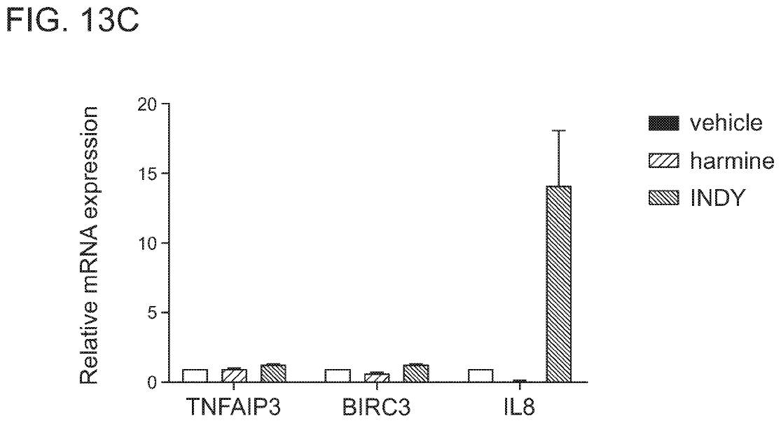

The finding that NF-κB is activated upon STAT3 inhibition suggests that the anti-cancer effects of pharmacologic STAT3 inhibitors may be limited by the compensatory upregulation of NF-κB transcriptional activity. On the other hand, it also raises the possibility that a combination of inhibitors targeting STAT3 and NF-κB might have enhanced efficacy. To identify inhibitors of NF-κB activity, the Prestwick chemical library was screened for specific NF-κB inhibitors using a cell-based NF-κB reporter assay. From this approach, harmine (FIG. 4A and FIG. 13A) was identified as an NF-κB inhibitor. Importantly, harmane, a structural variant of harmine, showed no effect on the transcriptional activity of NF-κB, suggesting that this is a pharmacologically specific effect.

To validate that harmine, but not harmane inhibits NF-κB activity, NF-κB-Luc expressing cells were pretreated with drug and then stimulated with TNF (FIG. 4B). Harmine, but not harmane, inhibited the activity of NF-κB dependent reporters, while not inhibiting STAT3 activity (FIG. 13A). Consistent with an on-target mechanism of action, it was identified that only harmine inhibited the viability of multiple myeloma cells that contain activated NF-κB, regardless of the activation status of STAT3 (FIG. 4C and FIG. 13B). Moreover, it was identified that harmine reduced the expression of endogenous NF-κB target genes (FIG. 4D).

To determine the mechanism by which harmine inhibits NF-κB activity, subcellular distribution of the p65 and p50 NF-κB subunits upon TNF stimulation was analyzed, and it was identified that harmine had no effect on the nuclear accumulation of these proteins (FIG. 4E). Dual-specificity tyrosine-regulated kinase-la (DYRK1 A) has been suggested to be a target of harmine (Gockler et al., 2009 FEBS J. 2009, 276(21):6324-37). To determine if DYRK1A had an effect on NF-κB activity, cells were treated with INDY, a DYRK1A inhibitor, and NF-κB target gene activity was measured. While harmine inhibited the expression of NF-κB target genes, INDY treatment did not inhibit their expression, and even increased expression of some NF-κB target genes, suggesting that DYRK1A is not involved in the inhibition of NF-κB by harmine (FIG. 13C). It was next determined whether harmine acted by inhibiting p65 binding to target gene promoters. Cells were pretreated with harmine or vehicle, stimulated with TNF, and ChIP to p65 at the NF-κB binding sites of target genes was performed. It was identified that harmine pretreatment reduced the binding of both p65 and RNA polymerase II upon TNF stimulation, suggesting a unique mechanism of action for this compound (FIG. 4F).

### Example 6: STAT3 and NF-κB Inhibitors have Synergistic Effects on Cancer Cells

Having validated that harmine is an inhibitor of NF-κB transcriptional function, it was next determined whether harmine prevented the upregulation of NF-κB target genes upon STAT3 inhibition. In fact, treatment of cells with harmine completely suppressed the induction of NF-κB target genes induced by the STAT3 inhibitor Jak inhibitor 1 (FIG. 5A). To determine if this combination of STAT3 and NF-κB inhibitors could synergize in killing cancer cells, multiple myeloma cells were treated with Jak inhibitor 1 to inhibit STAT3 and with harmine to inhibit NF-κB. It was identified that this combination led to synergistic cell killing (FIG. 5B) and enhanced apoptosis as measured by PARP cleavage (FIG. 5C). It was identified that additional STAT3 inhibitors also lead to enhanced killing when combined harmine (FIG. 5B). Thus, as described herein, combination treatment of cancer cells with STAT3 and NF-κB inhibitors is an important therapeutic option by preventing the compensatory activation of NF-κB that occurs when treating with STAT3 inhibitors alone.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of inhibiting nuclear factor kappa-light-chain-enhancer of activated B cells (NF-κB) function or activity in a cell comprising contacting the cell with an agent derived from Peganum harmala (Syrian rue), or an analogue thereof, thereby inhibiting NF-κB function or activity in a cell.

2. The method of claim 1, wherein the agent derived from Peganum harmala (Syrian rue) comprises harmine or harmol.

3. The method of claim 1, wherein the method further comprises administering infliximab, adalimumab, certolizumab pegol, golimumab, or etanercept.

4. The method of claim 1, wherein the NF-κB function or activity comprises NF-κB-dependent gene expression/transcriptional activity.

5. The method of claim 2, wherein the harmine inhibits expression of a NF-κB target gene selected from the group consisting of baculoviral TAP repeat-containing protein 3 (BIRC3), interleukin 8 (IL-8), and tumor necrosis factor alpha-induced protein 3 (TNFAIP3).

6. The method of claim 1, wherein the harmine is administered at a dose of 0.01 μM to 10 μM.

7. The method of claim 1, wherein NF-κB function or activity in the cell is inhibited by 10%-100%.

8. A method for treating or preventing a cancer or an inflammatory disease associated with aberrant NF-κB activity in a subject comprising:
administering to the subject a therapeutically effective amount of an agent derived from Peganum harmala (Syrian rue), or an analogue thereof, thereby treating or preventing the hyperproliferative disorder or inflammatory disease associated with aberrant NF-κB activity in the subject.

9. The method of claim 8, wherein the subject has been diagnosed with a hyperproliferative disorder or an inflammatory disease associated with aberrant NF-κB activity.

10. The method of claim 8, wherein the subject is identified as having elevated NF-κB activity, or wherein the subject is identified as in need of inhibiting NF-κB activity.

11. The method of claim 8, wherein the agent derived from Peganum harmala (Syrian rue) comprises harmine or harmol.

12. The method of claim 8, wherein the method further comprises administering infliximab, adalimumab, certolizumab pegol, golimumab, or etanercept.

13. The method of claim 8, wherein the NF-κB function or activity comprises NF-κB-dependent gene expression/transcriptional activity.

14. The method of claim 8, wherein the harmine inhibits expression of a NF-κB target gene selected from the group consisting of baculoviral inhibitor of apoptosis repeat-containing protein 3 (BIRC3), interleukin 8 (IL-8), and tumor necrosis factor alpha-induced protein 3 (TNFAIP3).

15. The method of claim 8, wherein the harmine is administered at a dose of 0.01 μM to 10 μM.

16. (canceled)

17. (canceled)

18. The method of claim 8, wherein the cancer is a solid tumor selected from the group consisting of esophageal cancer, breast cancer, melanoma, colon cancer, stomach or gastric cancer, ovarian cancer, pancreatic cancer, lung cancer, hepatic cancer, head and neck cancer, prostate cancer and brain cancer.

19. The method of claim 18, wherein the solid tumor comprises triple negative breast cancer or high grade serous ovarian cancer.

20. The method of claim 8, wherein the cancer comprises leukemia, lymphoma, or multiple myeloma, and wherein the leukemia or lymphoma is selected from the group consisting of acute lymphoblastic leukemia, acute myelogenous leukemia, chronic myelogenous leukemia, Hodgkin's disease, non-Hodgkin's lymphoma, T-cell lymphoma, B-cell lymphoma and chronic lymphocytic leukemia.

21. The method of claim 8, wherein the inflammatory disease associated with aberrant NF-κB activity comprises an autoimmune disease selected from the group consisting of celiac disease, diabetes mellitus type 1, Graves' disease, inflammatory bowel disease, multiple sclerosis, psoriasis, rheumatoid arthritis, and systemic lupus erythematosus.

22. (canceled)

23. (canceled)

24. The method of claim 8, further comprising administering
a chemotherapeutic agent selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and vinorelbine or
a signal transducer and activator of transcription 3 (STAT3) inhibitor selected from the group consisting of pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, 3-(2-hydroxyphenyl)-3-phenyl-N,N-dipropylpropanamide, and derivatives or analogues thereof.

25. (canceled)

26. An isolated ovarian cancer cell comprising a vector expressing a firefly luciferase reporter gene operably-linked to an NF-κB-dependent promoter.

27. The isolated ovarian cancer cell of claim 26, wherein the ovarian cancer cell comprises an OVCAR8 cell or an A2780 cell.

28. The isolated breast cancer cell of claim 26, wherein the cell comprises a vector expressing Renilla luciferase operably linked to a constitutive promoter.

29. A method of screening for a compound that inhibits NF-κB function and/or activity comprising:
providing one or more ovarian cancer cell(s) comprising a vector expressing a firefly luciferase reporter gene operably-linked to an NF-κB-dependent promoter;
and contacting the cell(s) with a candidate compound, wherein a decrease in the level of NF-κB-dependent luciferase activity in the presence of the candidate compound as compared to the level of NF-κB-dependent luciferase activity in the absence of the candidate compound indicates that the candidate compound inhibits NF-κB function and/or activity.

30. The method of claim 29, further comprising contacting the cell with an agent that induces the function and/or activity of NF-κB prior to contacting the cell with a candidate compound.

31. The method of claim 30, wherein the agent that induces the function and/or activity of NF-κB comprises TNFα.
